(19) United States
(12) Patent Application Publication
(10) Pub. No.: US 2026/0015609 A1
(43) Pub. Date: Jan. 15, 2026

(54) IMAGING-BASED HIGH-THROUGHPUT IDENTIFICATION OF BIOMOLECULES

(71) Applicant: YALE UNIVERSITY, New Haven, CT (US)

(72) Inventors: Joerg Bewersdorf, New Haven, CT (US); Florian Schüder, New Haven, CT (US); Brett Phelan, New Haven, CT (US)

(21) Appl. No.: 19/268,579

(22) Filed: Jul. 14, 2025

Related U.S. Application Data
(60) Provisional application No. 63/671,501, filed on Jul. 15, 2024.

(51) Int. Cl. C12N 15/10

(52) U.S. Cl. CPC C12N 15/1065

(57) ABSTRACT

Described herein is an imaging-based method for identifying RNA molecules in a sample, which is able to determine both the location and sequences of the RNA molecules. The method comprises: forming randomized barcode attached to the RNA molecules in the sample by sequential stepwise addition of permanent nucleic acid adapters randomly selected from a pool; acquiring imaging signals from the sequence-specific staining of the newly added permanent nucleic acid adapters after each addition of a permanent nucleic acid adapter, which links the location information of the RNA molecules with the barcoding; sequencing the barcoded RNA molecules, which links the sequences of the RNA molecules with the barcoding; and matching the location of the RNA molecules to the sequences thereof by matching the barcoding. Also described are kits for performing the method.

### CROSS-REFERENCE TO RELATED APPLICATIONS

The present application claims priority under 35 U.S.C. § 119 (e) to U.S. Provisional Patent Application No. 63/671,501, filed Jul. 15, 2024, which is incorporated herein by reference in its entirety.

### STATEMENT REGARDING FEDERALLY SPONSORED RESEARCH

This invention was made with government support under GM151829 awarded by the National Institutes of Health. The government has certain rights in the invention.

### BACKGROUND

Existing transcriptomics assays have various shortcomings. For example, array-based sequencing assays are able to determine mRNA sequences at a cellular scale, but have a hard time achieving subcellular resolutions. Fluorescence in situ hybridization (FISH) assays can study both the identity and spatial information of mRNA molecules and can achieve single molecule resolution, but cannot be used to study mRNA in a given cell en masse and can only detect mRNA where the sequence is known. In situ sequencing can also achieve single molecule resolution, but also suffers from low throughput.

There is a need for high-throughput assays that can extract both sequences and spatial information of large number of mRNA, as well as other molecules (e.g., other types of RNA molecules or DNA molecules) in a sample. The present study addresses this need.

### SUMMARY

In some aspects, the present invention is directed to the following nonlimiting embodiments:

### Method of Identifying RNA Molecules

In some aspects, the present invention is directed to a method of identifying RNA molecules in a sample.

In some embodiments, the method comprises forming a randomized barcode on each of a plurality of RNA molecules in the sample, which comprises the steps of:
attaching one by one in a stepwise manner, a plurality of permanent nucleic acid adapters to each of the plurality of RNA molecules, thereby forming a linear array of permanent nucleic acid adapters on each of the plurality of RNA molecules,
wherein each permanent nucleic acid adapter in the linear array is selected randomly from a pool of different permanent nucleic acid adapters, and
wherein each permanent nucleic acid adapter from the pool of different permanent nucleic acid adapters is associated with and identifiable by a detection motif.

In some embodiments, the method further comprises introducing detection motifs into the sample, thereby associating the permanent nucleic acid adapter newly added in the linear array with the corresponding detection motifs.

In some embodiments, the method further comprises acquiring imaging signals of the detection motifs in the sample.

In some embodiments, the method further comprises determining the location of the RNA molecules in the sample and identifying the newly added permanent nucleic acid adapter based on locations and types of the detected imaging signals of the detection motifs.

In some embodiments, the method further comprises combining the sequential arrangements of the permanent nucleic acid adapter in the linear arrays identified in the image acquisition steps into imaging-determined barcode information for each of the plurality of RNA molecules.

In some embodiments, the method further comprises sequencing the plurality of RNA molecules, as well as the barcodes attached thereto to obtain RNA sequence for each of the plurality of RNA molecules and sequencing-determined barcode information associated with each of the plurality of RNA sequence.

In some embodiments, the method further comprises matching the RNA sequences obtained in the sequencing step to the imaging signals of the detection motifs in the sample by matching the imaging-determined barcode information with the sequencing-determined barcode information, thereby determining the location of each of the plurality of RNA molecules and the sequence thereof.

In some embodiments, attaching the plurality of permanent nucleic acid adapters to each of the plurality of RNA molecules comprises, in each attaching step: introducing in the sample the pool of different permanent nucleic acid adapters.

In some embodiments, one permanent nucleic acid adapter selected from the pool of permanent nucleic acid adapters is added to the RNA molecules or the permanent nucleic acid adapter already attached to the plurality of RNA molecules in an immediate prior attaching step.

In some embodiments, each permanent nucleic acid adapter comprises:
a hybridization region that hybridizes with, in a permanent manner, the RNA molecules or the permanent nucleic acid adapter added to the RNA molecules in the immediate prior round of addition; and
one readout region selected from a predetermined group of readout regions, each associated with and identifiable by one detection motif.

In some embodiments, introducing the detection motifs into the sample allows each of the newly attached permanent nucleic acid adapter to bind to one corresponding detection motif in a reversible manner via the readout region thereof.

In some embodiments, the detection motifs reversibly bind to the corresponding permanent nucleic acid adapter via the bridging of a transient nucleic acid adapter.

In some embodiments, each detection motif is attached, optionally covalently, to a nucleic acid probe.

In some embodiments, the detection motifs are introduced into the sample together with a pool of transient nucleic acid adapters.

In some embodiments, each nucleic acid probe of the pool of transient nucleic acid adapters hybridizes with a corresponding transient nucleic acid adapter.

In some embodiments, each transient nucleic acid adapter hybridizes with the readout region of a corresponding permanent nucleic acid adapter in a reversible manner.

In some embodiments, the sample is a fixed sample.

In some embodiments, the plurality of RNA molecules are a plurality of mRNA molecules, and a first permanent nucleic acid adapter added in the first round of permanent nucleic acid adapter addition comprises: a hybridization region that hybridizes with, in a permanent manner, the poly(A) tail of the mRNA molecules.

In some embodiments, a dissociation constant (Kd) between the hybridization region of the permanent nucleic acid adapter and the plurality of RNA molecules, or a Kd between the hybridization region of the permanent nucleic acid adapter and the permanent nucleic acid adapter added in the immediate prior round of addition ranges from about 1000 fM to about 1 μM.

In some embodiments, an association rate constant (Kon) between the hybridization region of the permanent nucleic acid adapter and the RNA molecules, or a Kon between the hybridization region of the permanent nucleic acid adapter and the permanent nucleic acid adapter added in the immediate prior round of addition ranges from about 1*10³ 1/(M*sec)-1*10⁹ 1/(M*sec).

In some embodiments, a dissociation rate constant (Koff) between the hybridization region of the permanent nucleic acid adapter and the RNA molecules, or a Koff between the hybridization region of the permanent nucleic acid adapter and the permanent nucleic acid adapter added in the immediate prior round of addition ranges from about 1/2592000 1/sec to about 1/10 1/sec.

In some embodiments, a number of complementary base pairs between the hybridization region of the permanent nucleic acid adapter and the RNA molecules, or a number of complementary base pairs between the hybridization region of the permanent nucleic acid adapter and the permanent nucleic acid adapter added in the immediate prior round of addition ranges from about 3 nt to about 250 nt.

In some embodiments, a dissociation constant (Kd) between a permanent nucleic acid adapter and a corresponding transient adapter ranges from about 1 nM to about 1 mM.

In some embodiments, an association rate constant (Kon) between a permanent nucleic acid adapter and a corresponding transient adapter ranges from 1*10² 1/(M*sec) to about 1*10⁴ 1/(M*sec).

In some embodiments, a dissociation rate constant (Koff) between a permanent nucleic acid adapter and a corresponding transient adapter ranges from about 1/1000 (1/sec) to about 1 (1/sec).

In some embodiments, a number of complementary base pairs between a permanent nucleic acid adapter and a corresponding transient adapter ranges from about 6 nt about 10 nt.

In some embodiments, forming the randomized barcodes on plurality of RNA molecules in the sample comprises:
(a) introducing to the sample a pool of first permanent nucleic acid adapters, thereby attaching each of the plurality of RNA molecules with one random first permanent nucleic acid adapter selected from the pool, wherein each first permanent nucleic acid adapter is associated with and identifiable by a corresponding detection motif;
(b) introducing to the sample a pool of sense-strand permanent nucleic acid adapters, thereby attaching the plurality of RNA molecules with one random sense-strand permanent nucleic acid adapter selected from the pool, wherein each sense-strand permanent nucleic acid adapter is associated with and identifiable by a corresponding detection motif; and
(c) introducing to the sample a pool of antisense-strand permanent nucleic acid adapters, thereby attaching the plurality of RNA molecules with one random antisense-strand permanent nucleic acid adapter selected from the pool, wherein each antisense-strand permanent nucleic acid adapter is associated with and identifiable by a corresponding detection motif.

In some embodiments, step (b)-(c) are performed one or more times in this order after step (a).

In some embodiments, the randomized barcodes attached to each of the plurality of RNA molecules has the structure of: RNA-first permanent nucleic acid adapter-(antisense-strand permanent nucleic acid adapter-sense-strand permanent nucleic acid adapter) n, where n is an integer of 1 or larger.

In some embodiments, sequencing the plurality of RNA molecules and the barcodes attached thereto comprises: subjecting nucleic acid complexes formed by the plurality of RNA molecules added with the plurality of permanent nucleic acid adapters to reverse transcription and ligation to form a plurality of corresponding cDNA molecules; and sequencing the plurality of cDNA molecules.

In some embodiments, the detection motifs are a fluorescence motif, optionally a fluorescent protein, a fluorescent small molecule, or a quantum dot.

In some embodiments, the detection motifs are a metal nanoparticle, optionally a gold nanoparticle.

In some embodiments, the detection motifs are a Raman scattering motif, optionally a Raman dye, optionally a Raman dye suitable for a stimulated Raman scattering microscopy.

In some embodiments, the detection motifs are an isotope.

In some embodiments, the sample is washed after a step of attaching one permanent nucleic acid adapters to remove excessive permanent nucleic acid adapters from the sample.

In some embodiments, the sample is not washed after a step of attaching a one permanent nucleic acid adapters.

In some embodiments, the sample is washed after a step of image acquisition to remove detection motifs.

In some embodiments, the sample is not washed after a step of image acquisition.

In some embodiments, the sample is expanded according to an expansion microscopy technology.

In some embodiments, the method is not a method of identifying RNA molecules, but rather a method of identifying other types of molecules containing a nucleic acid sequence. In some embodiments, the method identifies DNA molecules or proteins.

In some embodiments, the method comprises:
(a) contacting the sample with a first plurality of permanent adapters, wherein each permanent adapter in the first plurality of permanent adapters comprising a poly-T region, a first handlebar region and a readout sequence;
(b) contacting the sample with a first plurality of transient adapters and a first plurality of imager probes, wherein
   each transient adapter in the first plurality of transient adapters comprises a region complementary to the readout sequence on at least one of the permanent adapters in the first plurality of permanent adapters and an imager probe docking site;
   each imager probe of the first plurality of imager probes comprises a region complementary to the detector docking site on at least one of the transient adapters in the first plurality of transient adapters and a detection motif;
(c) imaging the sample to detect the detection motifs in the first plurality of imager probes;
(d) contacting the sample with a second plurality of permanent adapters, wherein each permanent adapter in the second plurality of permanent adapters comprises a region complementary to the first handlebar region as well as at least one readout sequence, a second handlebar region and a readout sequence;
(e) contacting the sample with a second plurality of transient adapters and a second plurality of imager probes, wherein
   each transient adapter in the second plurality of transient adapters comprising a region complementary to the readout sequence on at least one of the permanent adapters in the second plurality of permanent adapters and an imager probe docking site;
   each imager probe of the second plurality of imager probes comprises a region complementary to the detector docking site on at least one of the transient adapter adapters in the second plurality of transient adapters and a detection motif;
(f) imaging the sample to detect the detection motifs in the second plurality of imager probes;
(g) contacting the sample with a third plurality of permanent adapters, wherein each permanent adapter in the third plurality of permanent adapters comprises a region complementary to the second handlebar region as well as at least one readout sequence, a first handlebar region and a readout sequence;
(h) contacting the sample with a third plurality of transient adapters and a third plurality of imager probes, wherein
   each transient adapter in the third plurality of transient adapters comprising a region complementary to the readout sequence on at least one of the permanent adapters in the third plurality of permanent adapters and an imager probes docking site;
   each imager probe of the third plurality of imager probes comprises a region complementary to the detector docking site on at least one of the transient adapters in the third plurality of transient adapters and a detection motif;
(i) imaging the sample to detect the detection motifs in the third plurality of imager probes;
(j) determining the spatial locations of the RNA molecules according to the spatial locations of the detection motifs detected in steps (c), (f), and/or (i);
(k) combining all detection motifs detected in steps (c), (f), and (i) into imaging-determined barcode information for the RNA molecules;
(l) sequencing nucleic acid complexes comprising the RNA molecules and the permanent adapters attached thereon to obtain RNA sequences correlated with sequencing determined barcode information;
(m) correlating the spatial locations of the RNA molecules with the RNA sequences by correlating the imaging-determined barcode information with the sequencing determined barcode information.

In some embodiments, steps (a), (c), (d), (f), (g), and (i) are carried out in this order.

In some embodiments, steps (d), (f), (g), (i) are repeated for one or more times before step (l).

In some embodiments, steps (b), (e) and (h) are carried out at the same time such that the first, second and third plurality of transient adapters and the first, second, and third plurality of imager probes are contacted with the sample as a mixture.

In some embodiments, steps (b), (e) and (h) are carried out before, after, or at the same time with step (a).

### Kit

In some aspects, the present invention is directed to a kit.

In some embodiments, the kit comprises: one or more pools of permanent nucleic acid adapters, wherein permanent nucleic acid adapters selected from the one or more pools of permanent nucleic acid adapters attach to RNA molecules one by one in a stepwise manner, thereby forming a barcode in the form of a linear array of permanent nucleic acid adapters.

In some embodiments, the kit further comprises a pool of detection motifs, wherein each detection motif of the pool of detection motifs associates specifically with a readout region of a permanent nucleic acid adapter from each of the one or more pools of permanent nucleic acid adapter.

In some embodiments, the one or more pools of permanent nucleic acid adapters comprises:
a pool of first permanent nucleic acid adapters;
a pool of antisense-strand permanent nucleic acid adapters; and
a pool of sense-strand permanent nucleic acid adapters.

In some embodiments, the permanent nucleic acid adapters from the pools of permanent nucleic acid adapters attach to RNA molecules one by one to form the barcode having the structure of: RNA-first permanent nucleic acid adapter-(antisense-strand permanent nucleic acid adapter-sense-strand permanent nucleic acid adapter) n, where n is an integer of 1 or larger.

In some embodiments, in the barcode, each one of the first permanent nucleic acid adapter, the antisense-strand permanent nucleic acid adapter, and the sense-strand permanent nucleic acid adapter is one permanent nucleic acid adapter selected from the corresponding pools of one permanent nucleic acid adapters.

In some embodiments, each first permanent nucleic acid adapter of the pool of first permanent nucleic acid adapters comprises: a hybridization region for permanently hybridizing with an RNA molecule; and a readout region associated with an identifiable by a corresponding detection motif.

In some embodiments, each antisense-strand permanent nucleic acid adapter of the pool of antisense-strand permanent nucleic acid adapters comprises: a hybridization region for permanently hybridizing with some or all of the first permanent nucleic acid adapters or for permanently hybridizing with some or all of the sense-strand permanent nucleic acid adapters; and a readout region associated with and identifiable by a corresponding detection motif.

In some embodiments, each sense-strand permanent nucleic acid adapter of the pool of sense-strand permanent nucleic acid adapters comprises: a hybridization region for permanently hybridizing with some or all of the antisense-strand permanent nucleic acid adapters; and a readout region associated with and identifiable by a corresponding detection motif.

In some embodiments, a dissociation constant (Kd) between the first permanent nucleic acid adapter and the RNA molecules being barcoded, or a Kd between the antisense-strand permanent nucleic acid adapter and the adjacent first permanent nucleic acid adapter or the adjacent sense-strand permanent nucleic acid adapter in the barcode ranges from about 1000 fM to about 1 μM.

In some embodiments, an association rate constant (Kon) between the first permanent nucleic acid adapter and the RNA molecules being barcoded, or a Kon between the antisense-strand permanent nucleic acid adapter and the adjacent first permanent nucleic acid adapter or the adjacent sense-strand permanent nucleic acid adapter in the barcode ranges from about 1*10³ 1/(M*sec) to about 1*10⁹ 1/(M*sec).

In some embodiments, a dissociation rate constant (Koff) between the first permanent nucleic acid adapter and the RNA molecules being barcoded, or a Koff between the antisense-strand permanent nucleic acid adapter and the adjacent first permanent nucleic acid adapter or the adjacent sense-strand permanent nucleic acid adapter in the barcode ranges from about 1/2592000 1/sec to about 1/10 1/sec.

In some embodiments, a number of complementary base pairs between the first permanent nucleic acid adapter and the RNA molecules being barcoded, or a Koff between the antisense-strand permanent nucleic acid adapter and the adjacent first permanent nucleic acid adapter or the adjacent sense-strand permanent nucleic acid adapter in the barcode ranges from about 3 nt to about 250 nt.

In some embodiments, each detection motif is attached, optionally covalently, to a nucleic acid probe, and the kit further comprises: a pool of transient nucleic acid adapters.

In some embodiments, each nucleic acid probe of the pool of transient nucleic acid adapters hybridizes with a corresponding transient nucleic acid adapter.

In some embodiments, each transient nucleic acid adapter hybridizes with the readout region of a corresponding permanent nucleic acid adapter in a reversible manner.

In some embodiments, a dissociation constant (Kd) between a permanent nucleic acid adapter and a corresponding transient adapter ranges from about 1 nM to about 1 mM.

In some embodiments, an association rate constant (Kon) between a permanent nucleic acid adapter and a corresponding transient adapter tor ranges from about 1*10² 1/(M*sec) to about 1*10⁴ 1/(M*sec).

In some embodiments, a dissociation rate constant (Koff) between a permanent nucleic acid adapter and a corresponding transient adapter ranges from about 1/1000 (1/sec) to about 1 (1/sec).

In some embodiments, a number of complementary base pairs between a permanent nucleic acid adapter and a corresponding transient adapter ranges from about 6 nt to about 10 nt.

In some embodiments, the detection motifs are a fluorescence motif, optionally a fluorescent protein, a fluorescent small molecule, or a quantum dot.

In some embodiments, the detection motifs are a metal nanoparticle, optionally a gold nanoparticle.

In some embodiments, the detection motifs are a Raman scattering motif, optionally a Raman dye, optionally a Raman dye suitable for a stimulated Raman scattering microscopy.

In some embodiments, the detection motifs are an isotope.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1E illustrate certain aspects of the RNA identification method, in accordance with some embodiments.

FIGS. 2A-2B illustrate certain aspects of the RNA barcoding method, in accordance with some embodiments.

FIG. 3 illustrates certain aspects of the detection of RNA molecule and the identification of barcoding elements attached to the RNA molecule, in accordance with some embodiments.

FIGS. 4A-4I illustrate certain aspects of the RNA identification method, in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In the study described herein, an imaging-based assay for the identification of RNA molecules in a sample, such as a cellular organelle, a cell, or a tissue, was developed.

According to the assay, RNA molecules in the sample are extended with multiple permanent nucleic acid adapters, which forms a linear array of permanent nucleic acid adapters. Each one of the permanent nucleic acid adapters in the linear array is selected randomly from a pool of permanent nucleic acid adapters. The random arrangements of the permanent nucleic acid adapters in the linear array in turn form unique barcodes attached to the RNA molecules.

Microscopy based imaging methods were developed which allow for each RNA molecule attached with a barcode to be visualized, as well as for the unique arrangement of permanent adapters within the barcode to be solved.

Sequencing methods were also developed to determine the sequences of the barcoded RNA molecules from the sample, as well as the sequences of the barcodes.

Since the imaging methods herein can create matches between RNA molecules visualized under microscopes and the barcodes attached thereto, and that the sequencing methods herein can create matches between sequences of RNA molecules and the barcodes attached thereto, the assay herein enables the determination of the sequences of RNA molecules visualized under a microscope, and such in a large-scale. In other words, the assay herein allows the detection and localization of part of or all of the RNA molecules within a sample, as well as the determination of the sequences of these RNA molecules.

Accordingly, in some aspects, the present invention is directed to a method of identifying RNA molecules in a sample.

Furthermore, the present study developed pools of nucleic acid adapters and detection motifs, such as fluorescence motifs, useful for the RNA identification methods herein.

Accordingly, in some aspects, the present invention is directed to a kit, such as a kit for performing the methods herein.

### Definitions

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### Method of Identifying Molecules in Samples

In some aspects, the present invention is directed to a method of identifying molecules in a sample.

In some embodiments, the molecules are RNA molecules, such as mRNA molecules. In some embodiments, the molecules are DNA molecules.

In some embodiments, the sample is a sample suitable for microscopic imaging study.

In some embodiments, the sample is a cellular organelle, a cell, or a tissue.

In some embodiments, the sample is a fixed sample such that RNA molecules within the sample do not move freely. In some embodiments, the biomolecules are cross-linked in the sample to restrict the movement of the biomolecules. Non-limiting examples of biomolecule cross-linking include formalin/formaldehyde cross-linking, glutaraldehyde cross-linking, dimethyl suberimidate (DMS) crosslinking, and the like. In some embodiments, the sample is dehydrated to restrict the movement of biomolecules. Non-limiting sample dehydration methods include ethanol fixation, methanol fixation, acetone fixation, and the like.

### Forming Randomized Barcodes on RNA Molecules

In some embodiments, the method comprises forming randomized barcodes on one or more RNA molecules in the sample.

In some embodiments, forming the randomized barcodes on one or more RNA molecules in the sample comprises: attaching a plurality of permanent nucleic acid adapters to each of the one or more RNA molecules one by one in a stepwise manner. In some embodiments, the plurality of permanent nucleic acid adapters forms a linear array that contains the barcoding information.

In some embodiments, each permanent nucleic acid adapter attached to the RNA molecules is randomly selected from one or more pools of different permanent nucleic acid adapters.

In some embodiments, each permanent nucleic acid adapter from the pool of different permanent nucleic acid adapters is associated with and identifiable by a detection motif.

In some embodiments, the term “permanent” means the permanent nucleic acid adapter binds to the molecule being imaged, or the previously attached permanent nucleic acid adapter for about 10 minutes or longer, about 20 minutes or longer, about 30 minutes or longer, about 1 hour or longer, about 2 hours or longer, or indefinitely under the experimental conditions. One of ordinary skill in the art would know how to design permanent nucleic acid adapters to ensure that the bindings are sufficiently long by taking into considerations the factors of, e.g., temperature, ionic strengths, and etc.

### Image Acquisition

In some embodiments, the method further comprises: introducing detection motifs into the sample, thereby associating the permanent nucleic acid adapter newly attached to the RNA molecules with the corresponding detection motifs.

In some embodiments, the method further comprises: detecting imaging signals of the detection motifs in the sample.

In some embodiments, the method further comprises: determining the location of the RNA molecules in the sample and identifying the permanent nucleic acid adapter attached in the most recent addition step based on the acquired imaging signals of the detection motifs.

### Imaging- and Sequencing-Determined Barcoding Information

In some embodiments, the method further comprises: combining the sequential arrangements of the permanent nucleic acid adapter identified in the image acquisition steps into imaging-determined barcode information for each of the one or more RNA molecules.

In some embodiments, the method further comprises: sequencing the one or more RNA molecules, as well as the barcodes attached thereto to obtain RNA sequences and sequencing-determined barcode information associated with the RNA sequences.

### Corresponding Sequences and Spatial Location

In some embodiments, the method further comprises: corresponding the obtained RNA sequences to the imaging signals of the one or more RNA molecules in the sample by matching the imaging-determined barcode information with the sequencing-determined barcode information.

### Permanent Nucleic Acid Adapters

In some embodiments, attaching the plurality of permanent nucleic acid adapters to each of the one or more RNA molecules comprises, in each attaching step: introducing in the sample the pool of different permanent nucleic acid adapters.

In some embodiments, one permanent nucleic acid adapter selected from the pool of permanent nucleic acid adapters is added to the RNA molecules or the permanent nucleic acid adapter already attached to the one or more RNA molecules in an immediate prior attaching step.

In some embodiments, each permanent nucleic acid adapter comprises: a hybridization region that hybridizes with, in a permanent manner, the RNA molecules or the permanent nucleic acid adapter added in the immediate prior round of addition; and one readout region selected from a predetermined group of readout regions, each associated with and identifiable by one detection motif.

In some embodiments, the RNA molecules are mRNA molecules, and a first permanent nucleic acid adapter added in the first round of permanent nucleic acid adapter addition comprises: a hybridization region that hybridizes with, in a permanent manner, the poly(A) tail of the mRNA molecules.

### Detection Motifs

In some embodiments, introducing the detection motifs into the sample allows each of the newly attached permanent nucleic acid adapters to bind to one corresponding detection motif in a reversible manner via the readout region thereof.

In some embodiments, the detection motifs reversibly bind to the corresponding permanent nucleic acid adapter via bridging of a transient nucleic acid adapter.

In some embodiments, each detection motif is attached, optionally covalently, to a nucleic acid probe, the detection motifs are introduced into the sample together with a pool of transient nucleic acid adapters, each nucleic acid probe of the pool of transient nucleic acid adapters hybridizes with a corresponding transient nucleic acid adapter, and each transient nucleic acid adapter hybridizes with the readout region of a corresponding permanent nucleic acid adapter in a reversible manner.

In some embodiments, the term “transient,” when referring to nucleic acid adapters, means that the nucleic acid adapters binds to one or more of their binding partners (e.g., the permanent adapters and/or the probe containing the detection motif) for about 10 minutes or less, about 5 minutes or less, about 2 minutes or less, about 1 minute or less, about 30 or less, about 10 seconds or less, about 5 second or less, about 2 second or less, or about 1 second or less, under the experimental conditions. One of ordinary skill in the art would know how to design transient nucleic acid adapters to ensure that the bindings are sufficiently short by taking into considerations the factors of, e.g., temperature, ionic strengths, and etc.

In some embodiments, the detection motifs are a fluorescence motif, optionally a fluorescent protein, a fluorescent small molecule, or a quantum dot.

In some embodiments, the detection motifs are a metal nanoparticle, optionally a gold nanoparticle.

In some embodiments, the detection motifs are a Raman scattering motif, optionally a Raman dye, optionally a Raman dye suitable for a stimulated Raman scattering microscopy.

In some embodiments, the detection motifs are an isotope.

### Permanent and Transient Hybridization

In some embodiments, the permanent hybridization between a permanent nucleic acid adapter and an RNA molecule, or the permanent hybridization between two permanent nucleic acid adapters means the hybridization is not disrupted by conditions (such as temperature, RNA/adapter concentrations, ion strength, etc.) involved in the method herein before the sample is destroyed for nucleic acid extraction.

In some embodiments, a dissociation constant (Kd) between the hybridization region of the permanent nucleic acid adapter and the RNA molecules, or a Kd between the hybridization region of the permanent nucleic acid adapter and the permanent nucleic acid adapter added in the immediate prior round of addition ranges from about 1000 fM to about 1 μM.

In some embodiments, an association rate constant (Kon) between the hybridization region of the permanent nucleic acid adapter and the RNA molecules, or a Kon between the hybridization region of the permanent nucleic acid adapter and the permanent nucleic acid adapter added in the immediate prior round of addition ranges from about 1*10⁹ 1/(M*sec) to about 1*10³ 1/(M*sec).

In some embodiments, a dissociation rate constant (Koff) between the hybridization region of the permanent nucleic acid adapter and the RNA molecules, or a Koff between the hybridization region of the permanent nucleic acid adapter and the permanent nucleic acid adapter added in the immediate prior round of addition ranges from about 1/2592000 1/sec to about 1/10 1/sec.

In some embodiments, a number of complementary base pairs between the hybridization region of the permanent nucleic acid adapter and the RNA molecules, or a number of complementary base pairs between the hybridization region of the permanent nucleic acid adapter and the permanent nucleic acid adapter added in the immediate prior round of addition ranges from about 3 nt to about 250 nt.

In some embodiments, the transient hybridization between a transient adapter and its permanent nucleic acid adapter binding partner is disrupted by and displaced with another permanent nucleic acid adapter that hybridizes with the previously referenced permanent nucleic acid adapter.

In some embodiments, a dissociation constant (Kd) between one permanent nucleic acid adapter and one corresponding transient adapter ranges from about 1 nM to about 1 mM.

In some embodiments, an association rate constant (Kon) between one permanent nucleic acid adapter and one corresponding transient adapter tor ranges from about 1*10⁴ 1/(M*sec) to about 1*10² 1/(M*sec).

In some embodiments, a dissociation rate constant (Koff) between one permanent nucleic acid adapter and one corresponding transient adapter ranges from about 1/1000 (1/sec) to about 1 (1/sec).

In some embodiments, a number of complementary base pairs between one permanent nucleic acid adapter and one corresponding transient adapter ranges from about 6 nt to about 10 nt.

### Three Adapter Pool Design

In some embodiments, the method herein uses three pools of permanent nucleic acid adapters: the first pool for directly hybridizing with the RNA molecules, the second pool for directly hybridizing with the first pool and the third pool, and the third pool for directly hybridizing with the second pool.

In some embodiments, forming the randomized barcodes on one or more RNA molecules in the sample comprises:
(a) introducing to the sample a pool of first permanent nucleic acid adapters, thereby attaching one or more RNA molecules with one first permanent nucleic acid adapter, wherein each first permanent nucleic acid adapter is associated with and identifiable by a corresponding detection motif;
(b) introducing to the sample a pool of sense-strand permanent nucleic acid adapters, thereby attaching the one or more RNA molecules with one sense-strand permanent nucleic acid adapter, wherein each sense-strand permanent nucleic acid adapter is associated with and identifiable by a corresponding detection motif; and
(c) introducing to the sample a pool of antisense-strand permanent nucleic acid adapters, thereby attaching the one or more RNA molecules with one antisense-strand permanent nucleic acid adapter, wherein each antisense-strand permanent nucleic acid adapter is associated with and identifiable by a corresponding detection motif.

In some embodiments, step (b)-(c) are performed one or more times in this order after step (a).

### Processing of Barcoded RNA Molecules for Sequencing

In some embodiments, sequencing the one or more RNA molecules and the barcodes attached thereto comprises: subjecting nucleic acid complexes formed by the one or more RNA molecules added with the plurality of permanent nucleic acid adapters to reverse transcription and ligation to form corresponding one or more cDNA molecules; and sequencing the one or more cDNA molecules.

### Sample Washing

In some embodiments, the sample is washed after a step of attaching one permanent nucleic acid adapters to remove excessive permanent nucleic acid adapters from the sample.

In some embodiments, the sample is not washed after a step of attaching a one permanent nucleic acid adapters.

In some embodiments, the sample is washed after a step of image acquisition to remove detection motifs.

In some embodiments, the sample is not washed after a step of image acquisition.

In some embodiments, the sample is expanded according to an expansion microscopy technology.

### Kit

In some aspects, the present invention is directed to a kit.

In some embodiments, the kit is a kit for identifying RNA molecules, such as mRNA molecules in a sample, such as a cellular organelle sample, a cell sample, a tissue sample, etc.

In some embodiments, the kit is a kit for performing the method described herein, such as in the “Method of Identifying RNA Molecules in Samples” section.

In some embodiments, the kit comprises: a pool of first permanent nucleic acid adapters; a pool of antisense-strand permanent nucleic acid adapters; a pool of sense-strand permanent nucleic acid adapters; and a pool of detection motifs.

In some embodiments, each detection motif of the pool of detection motifs associates specifically with a readout region of one first permanent nucleic acid adapter in the pool of first permanent nucleic acid adapters, one antisense-strand permanent nucleic acid adapter in the pool of antisense-strand permanent nucleic acid adapters, and/or one sense-strand permanent nucleic acid adapter in the pool of sense-strand permanent nucleic acid adapters. In some embodiments, the detection motifs are able to specifically associate with the readout regions in a sequence-specific manner.

In some embodiments, the permanent nucleic acid adapters from the pools of permanent nucleic acid adapters are capable of attaching to RNA molecules one by one to form a barcode having the structure of: RNA-first permanent nucleic acid adapter-(antisense-strand permanent nucleic acid adapter-sense-strand permanent nucleic acid adapter) n (n is an integer of 1 or larger), and each one of the first permanent nucleic acid adapter, the antisense-strand permanent nucleic acid adapter, and the sense-strand permanent nucleic acid adapter is one permanent nucleic acid adapter selected from the corresponding pools of one permanent nucleic acid adapters.

In some embodiments, each first permanent nucleic acid adapter of the pool of first permanent nucleic acid adapters comprises: a hybridization region for permanently hybridizing with an RNA molecule; and a readout region associated with and identifiable by a corresponding detection motif.

In some embodiments, each antisense-strand permanent nucleic acid adapter of the pool of antisense-strand permanent nucleic acid adapters comprises: a hybridization region for permanently hybridizing with some or all of the first permanent nucleic acid adapters or for permanently hybridizing with some or all of the sense-strand permanent nucleic acid adapters; and a readout region associated with and identifiable by a corresponding detection motif.

In some embodiments, each sense-strand permanent nucleic acid adapter of the pool of sense-strand permanent nucleic acid adapters comprises: a hybridization region for permanently hybridizing with some or all of the antisense-strand permanent nucleic acid adapters; and a readout region associated with and identifiable by a corresponding detection motif.

In some embodiments, a dissociation constant (Kd) between the first permanent nucleic acid adapter and the RNA molecules in the barcode, or a Kd between the antisense-strand permanent nucleic acid adapter and the adjacent first permanent nucleic acid adapter or sense-strand permanent nucleic acid adapter in the barcode ranges from about 1000 fM to about 1 μM.

In some embodiments, an association rate constant (Kon) between the first permanent nucleic acid adapter and the RNA molecules in the barcode, or a Kon between the antisense-strand permanent nucleic acid adapter and the adjacent first permanent nucleic acid adapter or sense-strand permanent nucleic acid adapter in the barcode ranges from about 1*10⁹ 1/(M*sec) to about 1*10³ 1/(M*sec).

In some embodiments, a dissociation rate constant (Koff) between the first permanent nucleic acid adapter and the RNA molecules in the barcode, or a Koff between the antisense-strand permanent nucleic acid adapter and the adjacent first permanent nucleic acid adapter or sense-strand permanent nucleic acid adapter in the barcode ranges from about 1/2592000 1/sec to about 1/10 1/sec.

In some embodiments, a number of complementary base pairs between the first permanent nucleic acid adapter and the RNA molecules in the barcode, or a Koff between the antisense-strand permanent nucleic acid adapter and the adjacent first permanent nucleic acid adapter or sense-strand permanent nucleic acid adapter in the barcode ranges from about 3 nt to about 250 nt.

In some embodiments, each detection motif is attached, optionally covalently, to a nucleic acid probe. In some embodiments, the kit further comprises: a pool of transient nucleic acid adapters. In some embodiments, each nucleic acid probe of the pool of transient nucleic acid adapters hybridizes with a corresponding transient nucleic acid adapter. In some embodiments, each transient nucleic acid adapter hybridizes with the readout region of a corresponding permanent nucleic acid adapter in a reversible manner.

In some embodiments, a dissociation constant (Kd) between one permanent nucleic acid adapter and one corresponding transient adapter ranges from about 1 nM to about 1 mM.

In some embodiments, an association rate constant (Kon) between one permanent nucleic acid adapter and one corresponding transient adapter tor ranges from about 1*10² 1/(M*sec)-1*10⁴ 1/(M*sec).

In some embodiments, a dissociation rate constant (Koff) between one permanent nucleic acid adapter and one corresponding transient adapter ranges from about 1/1000 (1/sec) to about 1 (1/sec).

In some embodiments, a number of complementary base pairs between one permanent nucleic acid adapter and one corresponding transient adapter ranges from about 6 to about 10.

In some embodiments, the detection motifs are a fluorescence motif, optionally a fluorescent protein, a fluorescent small molecule, or a quantum dot.

In some embodiments, the detection motifs are a metal nanoparticle, optionally a gold nanoparticle.

In some embodiments, the detection motifs are a Raman scattering motif, optionally a Raman dye, optionally a Raman dye suitable for a stimulated Raman scattering microscopy.

In some embodiments, the detection motifs are an isotope.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

It should be noted that, although the Example section describes the method herein in reference to the identification and location of mRNA molecules, the method herein is not limited thereto. The method herein can be used to study any types of nucleic acid molecules, or molecules to which nucleic acid molecules can be attached, in an untargeted manner. For example, to study micro-RNA molecules, a poly-A region can be added by use of a polyadenylating enzyme, following which the protocol would be as describes as above. Additionally, by adding nucleic acid strands to proteins via covalent attachment and designing the pool of the first permanent nucleic acid adapters to have a region complementary to these sequences, the method described herein can be thus adapted to localize these proteins and identify them via an appropriate sequencing method which captures the permanent nucleic acid adapter sequence and corresponding protein identity in a paired manner.adapter

### Example 1: Overall Method

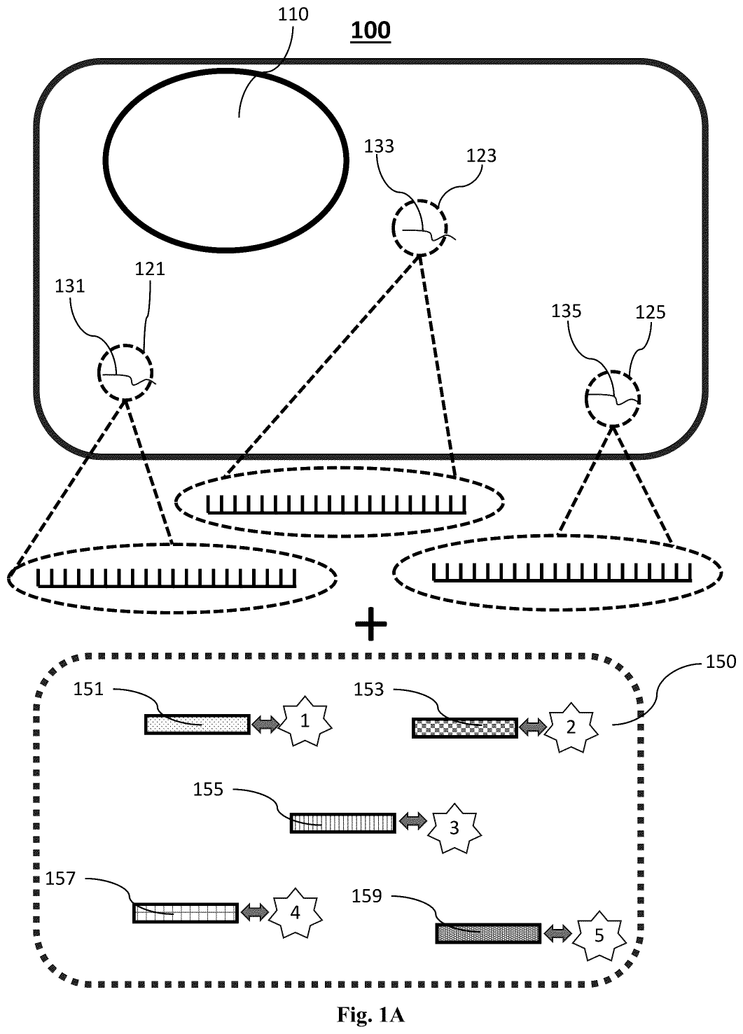

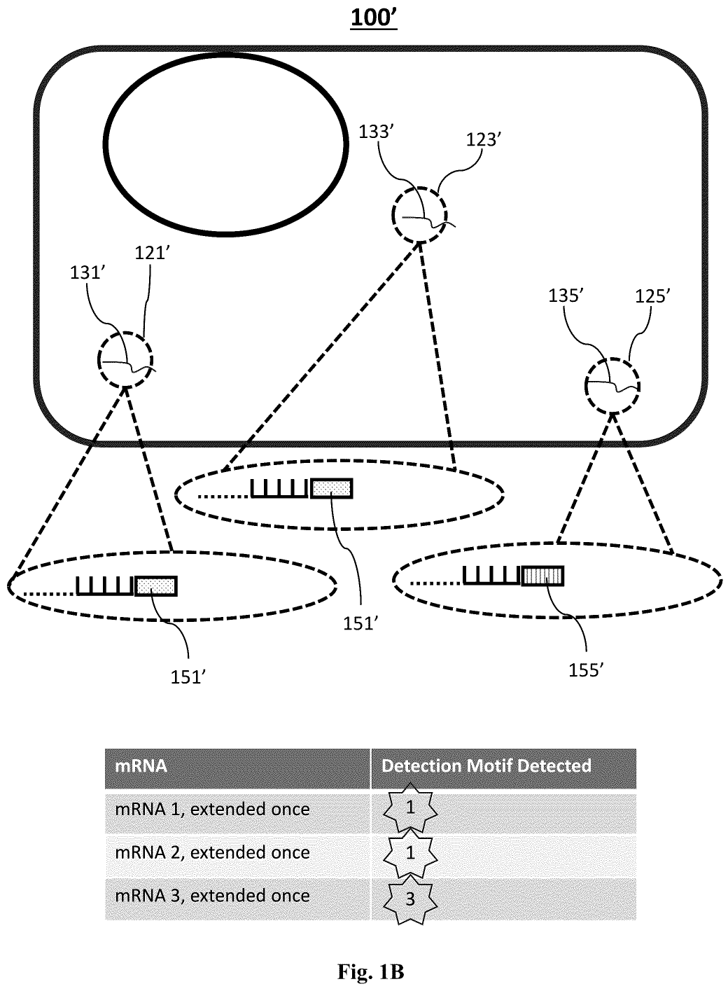

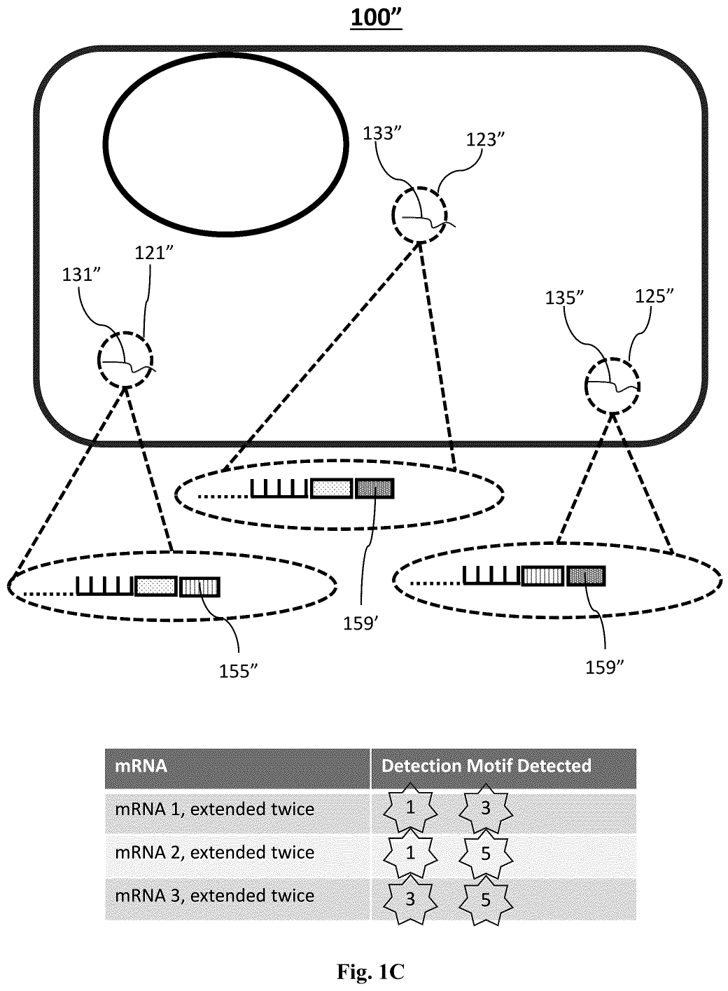

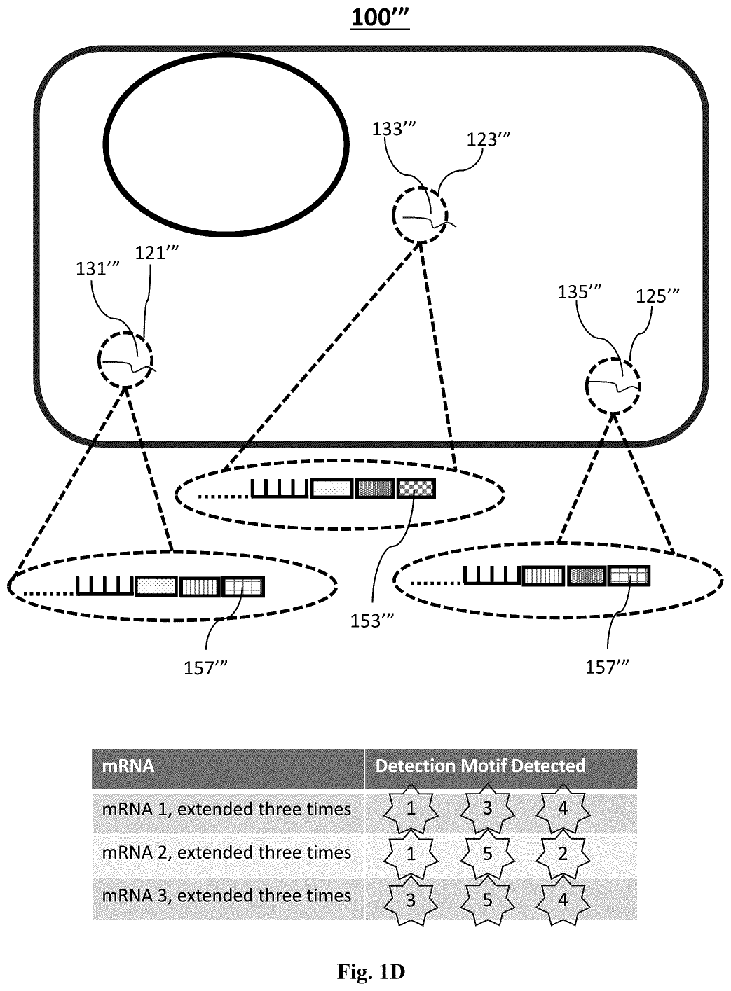

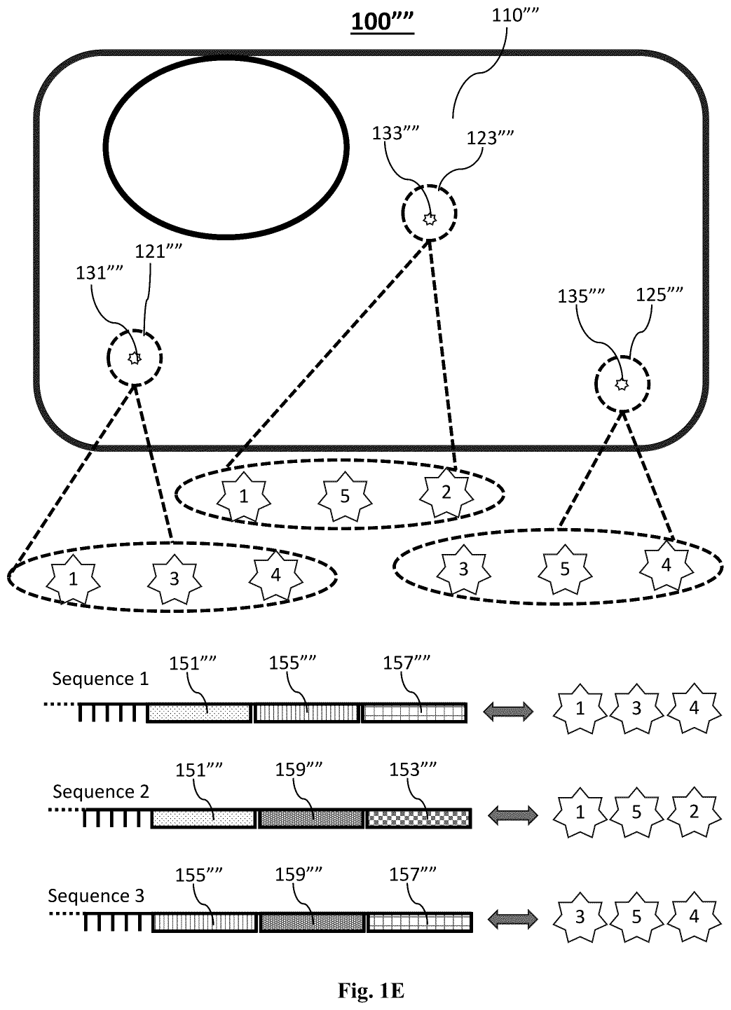

Referring to the FIGS. 1A-1E, mRNA molecules in a cell sample are identified and located by the method herein in accordance with some embodiments.

Referring to FIG. 1A, a cell sample 100 comprising a nucleus 110 is fixed to prevent the RNA molecules in the cell from moving freely. The cell sample 100 comprises three mRNA molecules—a first mRNA molecule 131 located in location 121, a second mRNA molecule 133 located in location 123, and a third mRNA molecule 135 located in location 125.

To identify the three mRNA molecules in the cell sample 100, the mRNA molecules is subjected to a several rounds of additions of permanent nucleic acid adapters, which forms barcodes on the mRNA molecules in the form of linear arrays.

Referring to FIG. 1A, a pool of first permanent nucleic acid adapters 150 is introduced in the cell sample. The pool of first permanent nucleic acid adapters 150 comprises a first permanent nucleic acid adapter-1 151, which is associated with and identifiable by the detection motif 1; a first permanent nucleic acid adapter-2 153, which is associated with and identifiable by the detection motif 2; a first permanent nucleic acid adapter-3 155, which is associated with and identifiable by the detection motif 3; a first permanent nucleic acid adapter-4 157, which is associated with and identifiable by the detection motif 4; and a first permanent nucleic acid adapter-5 159, which is associated with and identifiable by the detection motif 5.

As an example, the five detection motifs in FIG. 1A can be five different fluorescence motifs such as five different fluorescence proteins and/or quantum dots that emit light of different wavelengths. The detection motifs can also include different nucleic acid sequences so that each detection motif only hybridizes, either directly or indirectly, with only one of the five first permanent nucleic acid adapters within the pool.

In each of the three mRNA molecules, one random first permanent nucleic acid adapter from the pool 150 is added, thereby extending the mRNA molecules once. Since the matching between the first permanent nucleic acid adapters in the pool and the mRNA molecules is a random process, which permanent nucleic acid adapter is attached to each of the mRNA molecules cannot be controlled or predicted. However, because each of the first permanent nucleic acid adapters is associated with and identifiable with only one detection motif, such information can be deduced by detecting the detection motifs.

Referring to FIG. 1B, the signals of the detection motif 1 are detected in location 121′, where the once extended first mRNA molecule 131′ locates, and location 123′, where the once extended second mRNA molecule 133′ locates. The signal of the detection motif 3 is detected in location 125′, where the once extended third mRNA molecule 135′ locates. Based on the known correlations between the first permanent nucleic acid adapters and the detection motifs, it can be deduced that the once extended first mRNA molecule 131′ now comprises the first permanent nucleic acid adapter-1 151′, the once extended second mRNA molecule 133′ now comprises the first permanent nucleic acid adapter-1 151′, and the once extended third mRNA molecule 135′ now comprises the first permanent nucleic acid adapter-3 155′.

The same steps shown in FIGS. 1A-1B are then repeated two more times to attach two additional permanent nucleic acid adapters: one second permanent nucleic acid adapter selected randomly from a pool of second permanent nucleic acid adapter, and one third permanent nucleic acid adapter selected randomly from a pool of third permanent nucleic acid adapter.

Referring to FIG. 1C, the once extended mRNA molecules are subjected to a second round of permanent nucleic acid adapter addition to generate twice extended mRNA molecules in the sample 100″. In FIG. 1C, detection motif 3 is detected in the first location 121″, and detection motif 5 is detected in the second and third locations 123″ and 125″. It can thus be deduced that, in this round of addition, the second permanent nucleic acid adapter-3 155″ is attached to the first mRNA molecule 131″, and the second permanent nucleic acid adapter-5 159″ is attached to both the second mRNA molecule 133″ and the third mRNA molecule 135″.

Referring to FIG. 1D, the twice extended mRNA molecules are subjected to a third round of permanent nucleic acid adapter addition to generate three-times extended mRNA molecules in the sample 100′″. In FIG. 1D, detection motif 4 is detected in the first location 121″′ and the third location 125′″, and detection motif 5 is detected in the second location 123′″. It can thus be deduced that, in this round of addition, the third permanent nucleic acid adapter-4 157′″ is attached to the first mRNA molecule 131″ and the third mRNA molecule 135′″, and the third permanent nucleic acid adapter-2 153′″ is attached to the second mRNA molecule 133′″.

Referring to FIG. 1E, now that it is known that the first mRNA molecule 131″″ located at location 121″″ is associated with detection motif combination 1-3-4 (and therefore the permanent nucleic acid adapters 1-3-4), the second mRNA molecule 133″″ located at location 123″″ is associated with detection motif combination 1-5-2 (and therefore permanent nucleic acid adapters 1-5-2), and the third mRNA molecule 135″″ located at location 125″″ is associated with detection motif combination 3-5-4 (and therefore permanent nucleic acid adapters 3-5-4), all the mRNA molecules added with the permanent adapters in the cell sample 100″″ can be extracted, processed, and sequenced. For example, each mRNA molecule and the permanent adapters attached thereon can then processed into a continuous nucleic acid strand, and sequenced.

The sequence results are three different nucleic acid sequences, which can be attributed to one of the three mRNA molecules based on the permanent adapter sequence information contained therein:
One of the three sequences contains mRNA sequence 1, as well as the sequences of the first permanent nucleic acid adapter-1 (151″″), the second permanent nucleic acid adapter-3 (155″″), and the third permanent nucleic acid adapter-4 (157″″), arranged in this order. It can thus be deduced that Sequence 1 is the sequence of the first mRNA 131″″ located at location 121″″;
One of the three sequences contains mRNA sequence 2, as well as the sequences of the first permanent nucleic acid adapter-1 (151″″), the second permanent nucleic acid adapter-5 (159″″), and the third permanent nucleic acid adapter-2 (153″″), arranged in this order. It can thus be deduced that Sequence 2 is the sequence of the second mRNA 133″″ located at location 123″″; and
One of the three sequences contains mRNA sequence 3, as well as the sequences of the first permanent nucleic acid adapter-3 (155″″), the second permanent nucleic acid adapter-5 (159″″), and the third permanent nucleic acid-4 (157″″), arranged in this order. It can thus be deduced that Sequence 3 is the sequence of the third mRNA 135″″ located at location 125″″.

Accordingly, performing the steps illustrated in FIGS. 1A-1E allowed both the locations and the sequences of all the three mRNA molecules in the cell sample determined.

It should be noted that additional rounds of mRNA extension/permanent nucleic acid adapter adding can be performed to increase the number of possible random barcodes. It should also be noted that the pool of the second permanent nucleic acid adapters and the pool of the second permanent nucleic acid adapters can be re-applied in alternative after the three rounds extension.

In the oversimplified scenario of illustrated in FIGS. 1A-1E, three rounds of extension with pools each containing five different permanent adapter-detection motif pair is sufficient. The reason is that the cell sample only contains three mRNA molecules, and the three rounds of adapter additions (each selected from a pool of five different permanent adapter-detection motif pair) can generate 5×5×5=125 possible combinations, which are close to 2 orders of magnitude higher than the total numbers of mRNA molecules in the sample. As such, the chance of any given two different mRNA molecules being attached with the same barcode is sufficiently low.

mRNA numbers in real cells are significantly higher than illustrated in FIGS. 1A-1E. For example, the average number of mRNA molecules in a mammalian cell is about 3.6×10⁵. As such, a possible combination of permanent adapters in the barcode can be 10⁷ or more in order to sufficiently cover all the mRNA molecules in one cell, which is quite feasible according to the methods herein. For example, if 10 different permanent adapter/detection motif pair are used in each addition step, only seven rounds of permanent adapter additions would be sufficient to generate the needed number of combinations to sufficiently cover all mRNA molecules in the cell. For another example, if 6 different permanent adapter/detection motif pair are used in each addition step, only nine rounds of permanent adapter additions would be sufficient to generate the needed number of combinations to sufficiently cover all mRNA molecules in the cell (6⁹≈10⁷).

### Example 2: MRNA Barcoding, and Barcoded mRNA Processing

In Example 2, the steps by which permanent nucleic acid adapters are added to the mRNA molecules to form the barcode, as well as the processing of the barcoded mRNA molecules for sequencing, are exemplified.

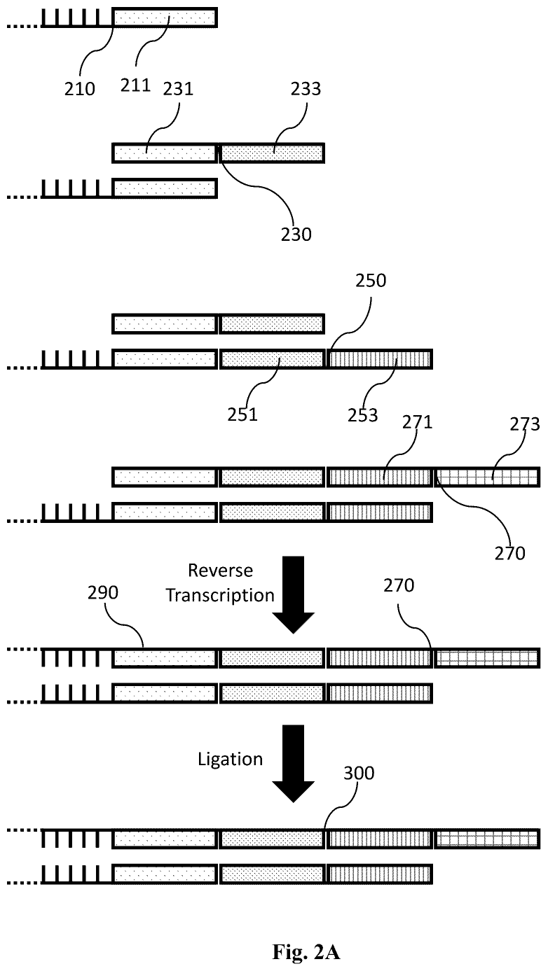

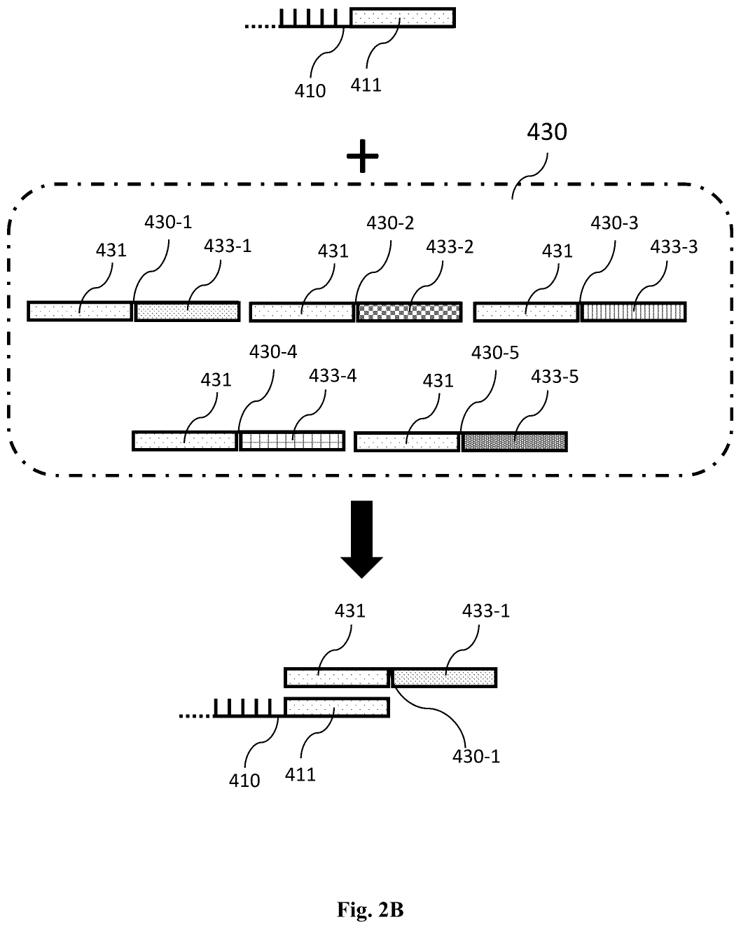

Referring to FIG. 2A, mRNA molecule 210 comprises a poly(A) tail 211 at a 3′-terminal end thereof. A pool of first permanent nucleic acid adapters 230, which comprises a poly(A) hybridization region 231 (e.g., a poly(T) sequence) and a readout region 233, is contacted with the mRNA molecule 210.

Referring to FIG. 2B, the pool of first permanent nucleic acid adapter 430 comprises five first permanent nucleic acid adapters (430-1 to 430-5) having the same hybridization region 431 for hybridizing with the mRNA molecule, but different readout regions (433-1 to 433-5) that are associated with and identifiable by different detection motifs. Each of the readout regions is associated with and identifiable by one detection motif.

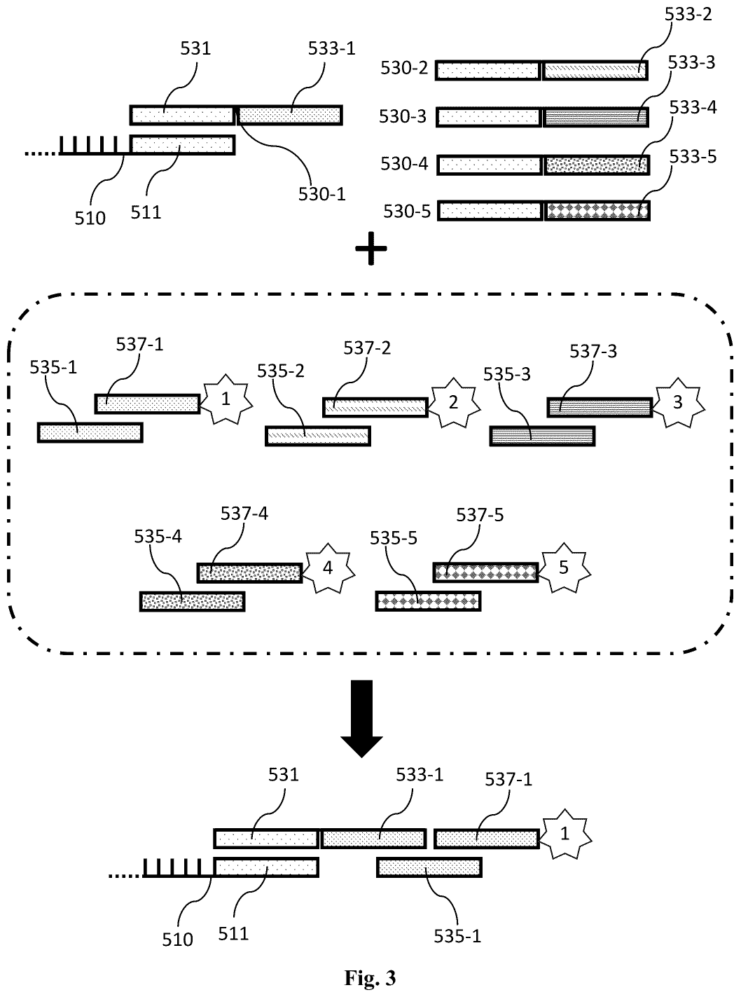

Due to the lack of selectivity in the hybridization between the hybridization region 431 and the poly(A) tail 411, a random first permanent nucleic acid adapter from the pool 430 is added to the mRNA molecule 410 via the hybridization between the poly(A) tail 411 and the first hybridization region 431. After the addition of the first permanent nucleic acid adapter to the mRNA, imaging techniques are used to determine which first permanent nucleic acid adapter from the pool 430 is attached to the mRNA molecule (see e.g., Example 3 and FIG. 3 for description of the imaging techniques). The imaging results reveal that the first permanent nucleic acid adapter 430-1 is added onto the mRNA molecule 410.

Referring back to FIG. 2A, after the addition of the first permanent nucleic acid adapter 230, a pool of second permanent nucleic acid adapters 250 and a pool of third permanent nucleic acid adapters 270 are added to the sample in two different addition steps.

Each second permanent nucleic acid adapter from the pool of second permanent nucleic acid adapters 250 comprises a second hybridization region 251, which hybridizes with the first readout region 233 of the first permanent nucleic acid adapter 230, as well as a second readout region 253 for associating with detection motifs.

Since the first readout region 233 of the first permanent nucleic acid adapter pool 230 is a pool of different readout regions, the second hybridization region 251 is also a pool of different hybridization regions, capable of hybridizing with each first readout region 233 within the first permanent nucleic acid adapter pool 230. For example, if the first permanent nucleic acid adapter pool 230 comprises five different first permanent nucleic acid adapters, each having a unique first readout region, the pool of second permanent nucleic acid adapters 250 can include a pool of five second hybridization regions, one for hybridizing with each first readout region. If the pool of second permanent nucleic acid adapters 250 also have a pool of five second readout regions, a total of 25 (5×5) different second permanent nucleic acid adapters is needed to form the second pool.

The design and application of the third pool of permanent nucleic acid adapter 270 is similar to that of the second pool of permanent nucleic acid adapter 250, and will not be detailed here.

It should be noted that, while the first pool 230 can only be used to extend the mRNA molecules once, the second pool 250 and the third pool 270 can be used in an alternative manner in further rounds of mRNA extension to increase the complexity of the barcodes attached to the mRNA molecules. The second pool 250 can be used to extend the mRNA molecules in the antisense strand, and the third pool 270 can be used to extend the mRNA molecules in the sense strand.

After all the mRNA extension steps have been carried out and images have been acquired, the nucleotide complex comprising the mRNA 210, and the first, second, and third permanent nucleic acid adapters 230, 250 and 270 is subjected to reverse transcription. In the reverse transcription step, the reverse transcriptase uses the first permanent nucleic acid adapter 230 as the primer and the mRNA molecule 210 as the template to reverse transcribe the mRNA molecule 210, thereby forming the reverse strand cDNA 290 of the mRNA molecule 210. The reverse strand cDNA 290 and the adjacent third permanent nucleic acid adapter 270 are then ligated together to form one single strand 300, which can be sequenced. Sequencing the nucleotide strand 300 will reveal the sequence of the mRNA molecule 210, as well as the added barcode attached to the mRNA (permanent nucleic acid adapters 230, 250 and 270). The barcoding information can be used to correlate the sequence of the mRNA molecule 210 to the detection motif signals detected during the imaging process of the sample, which reveals the location of the mRNA molecules in the sample.

### Example 3: Imaging-Based Identification of Barcoding Adapters

In Example 3, the microscopic imaging-based identification of barcoding permanent adapters newly attached to the mRNA molecules after each mRNA extension is exemplified.

Referring to FIG. 3, the identification process is exemplified with the pool of first permanent nucleic acid adapters added in the first round mRNA extension.

In FIG. 3, a sample containing an mRNA molecule 510 (which is immobilized by sample fixation) is introduced with a pool of the first permanent nucleic acid adapter (530-1 to 530-5), each comprising the same first hybridization region 531 for hybridizing with any mRNA molecules, but different first readout regions (533-1 to 533-5) associated with different detection motifs (detection motifs 1 to 5).

By a random selection from the pool of first permanent nucleic acid adapters (530-1 to 530-5), the first permanent nucleic acid adapter-1 530-1 is hybridized with the mRNA molecule 510. That which first permanent nucleic acid adapter is added onto the mRNA molecule 510 is not readily known. To extract that information, a pool of transient adapters (535-1 to 535-5) and imager probes (537-1 to 537-5, which comprising detection motifs 1 to 5, respectively) is added to the sample. Each pair of transient adapter and imager probe hybridizes exclusively with only one of the five first permanent nucleic acid adapters. Since only the detection motif 1 on the imager probe 537-1 can bind to the first permanent nucleic acid adapter 530-1 added to the mRNA molecule 510 (via the bridging of the transient adapter 535-1), only detection motif 1 is captured by the mRNA molecule 510. Since mRNA molecule 510 is immobilized, detection motif 1 is immobilized indirectly, as well, and generates a signal when visualized by microscopy methods. The detection motifs that do not immobilized by mRNA molecule 510 are either immobilized by other mRNA molecules and generate signals elsewhere, or move freely in the sample. Detection motifs that move freely in the sample do not generate microscopic signals, as the signals are dispersed into background noise.

As such, the transient adapter 535-1 mediated association between the first permanent nucleic acid adapter 510 and the detection motif 1 not only provides a microscopic signal for the mRNA molecule 510, but also identifies the first permanent nucleic acid adapter attached to the mRNA molecule 510.

The determination of the permanent nucleic acid adapters added to the mRNA molecule in the subsequent rounds are carried out in similar manners.

### Example 4

Example 4 provides another variation of the method herein in the identification and location of mRNA molecules in a sample.

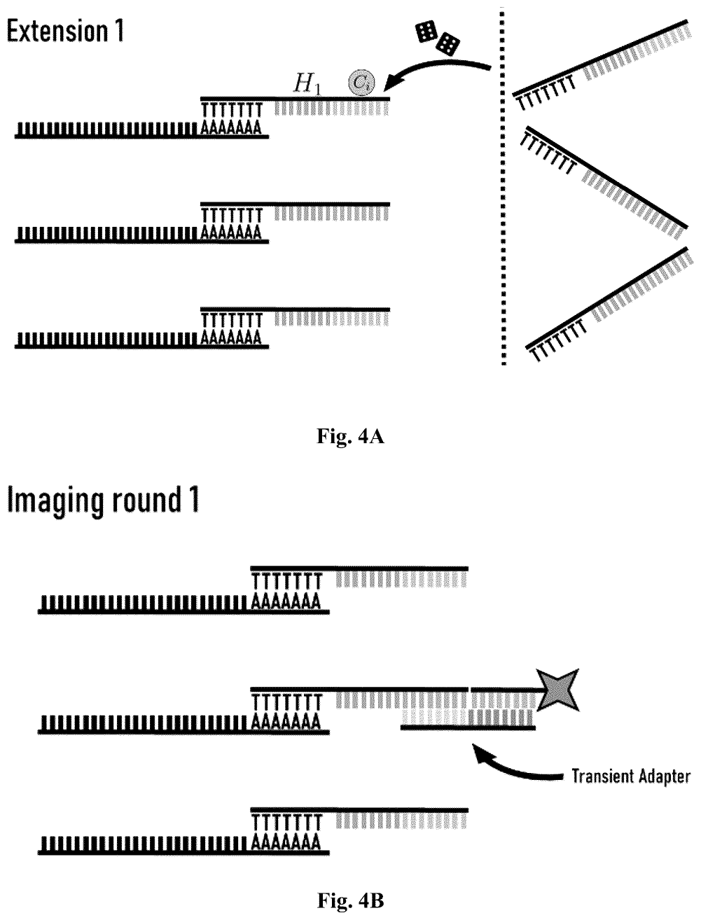

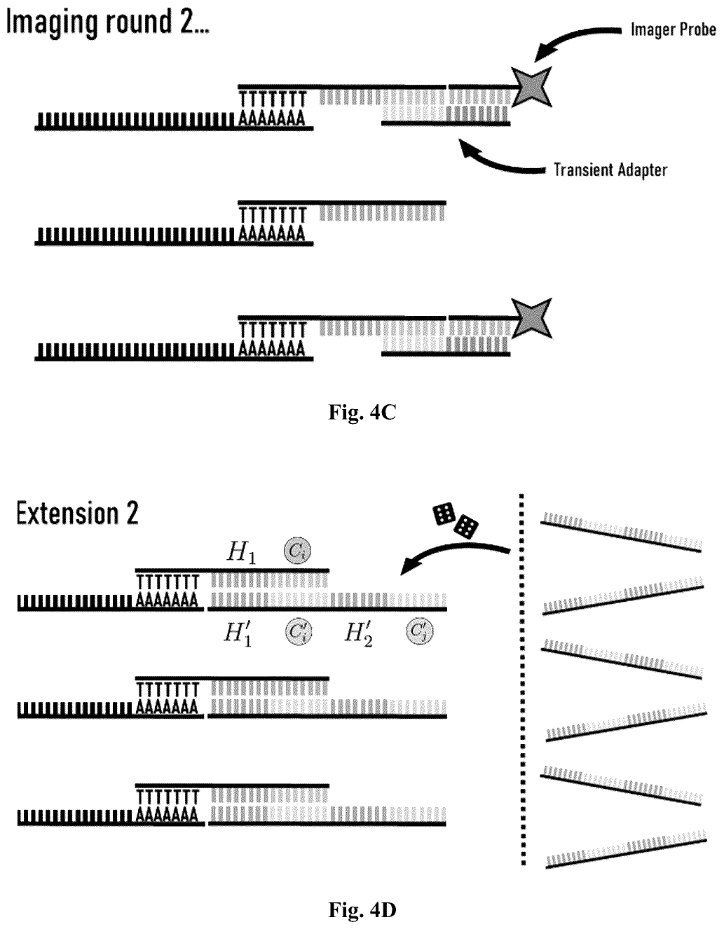

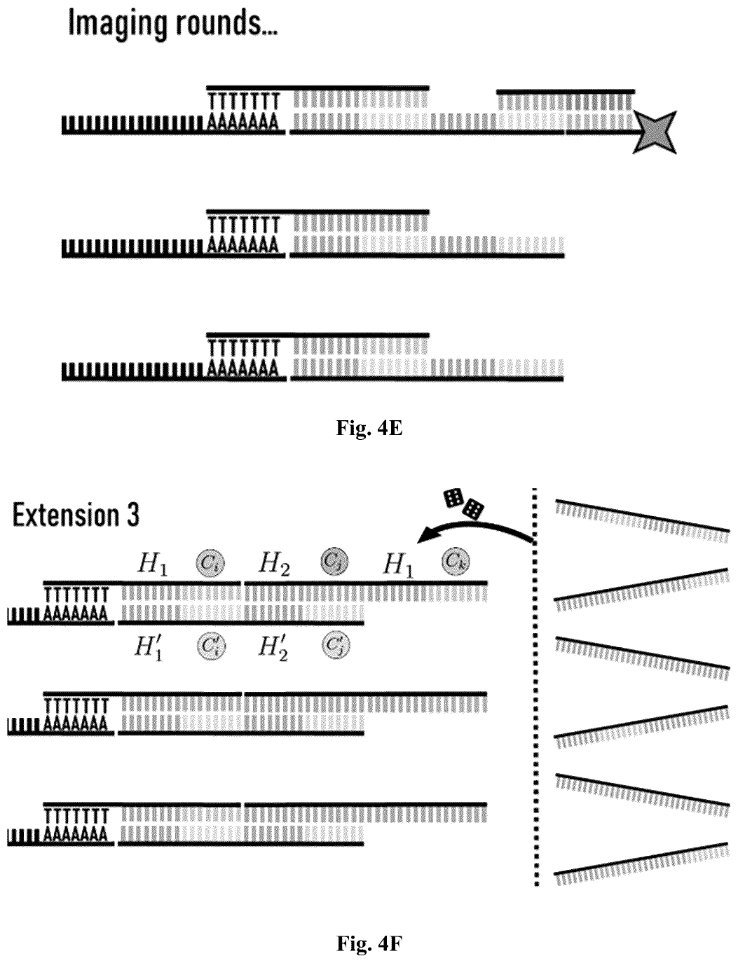

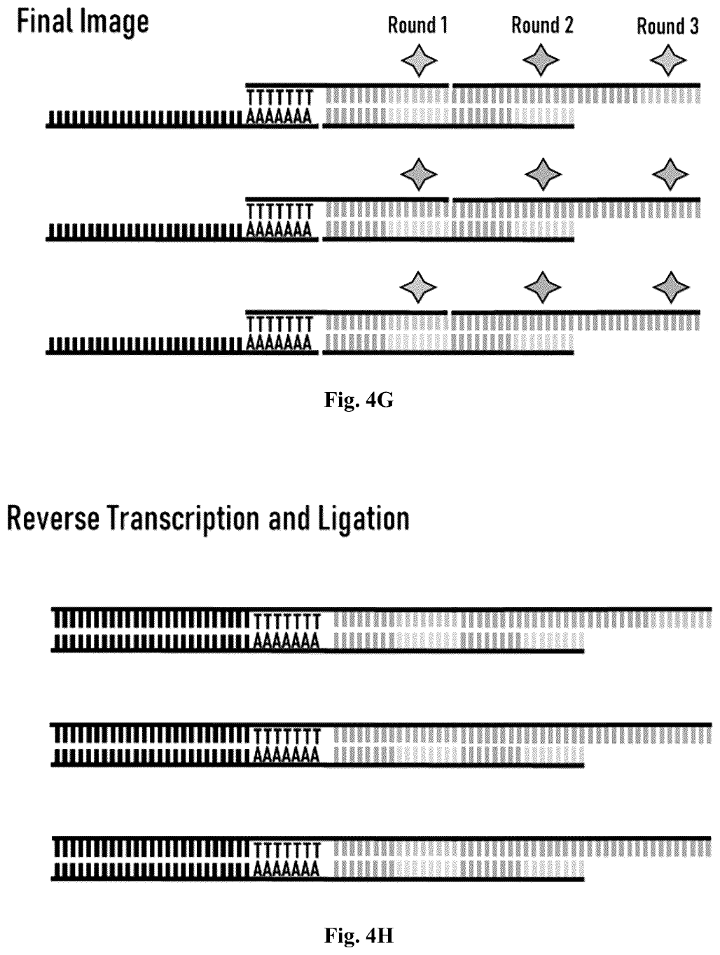

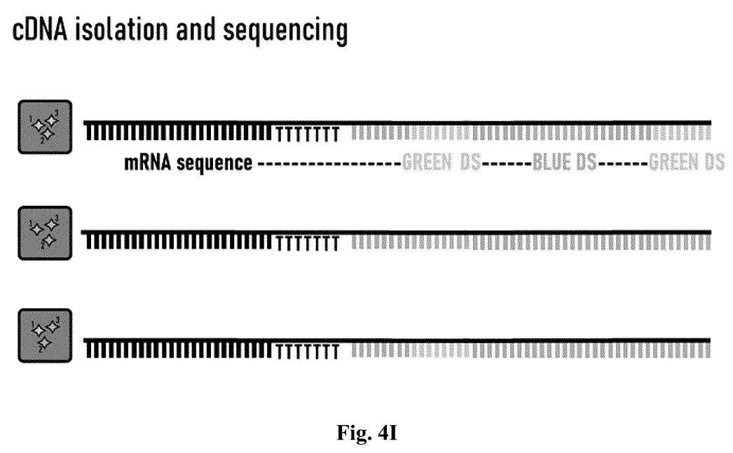

Referring to FIG. 4A, in a first rounds of mRNA extension, a pool of first permanent nucleic acid adapters are added to the sample. The first permanent nucleic acid adapters bind the poly-A tail of mRNA with a complementary poly-T region and exposing a random FLASH-PAINT docking site.

Referring to FIGS. 4B-4C, orthogonal FLASH-PAINT adapters are added sequentially in batches of size equal to the number of simultaneously uniquely identifiable detection motifs (such as spectrally distinct fluorescent molecules) until all orthogonal docking sites are imaged.

Referring to FIG. 4D, in the second round of mRNA extension, second permanent nucleic acid adapters are added. These adapters bind the previous round's handle sequence (H1) and FLASH-PAINT docking site and expose a new, orthogonal handle (H2′) and reverse complement of a random FLASH-PAINT docking sites. This ensures permanent binding in the desired orientation and prevents self-binding between any pair of permanent nucleic acid adapters in the second round.

Referring to FIG. 4E, FLASH-PAINT imaging is repeated as before to obtain an image of each orthogonal docking site.

Referring to FIG. 4F, in the next round of mRNA extension, third permanent nucleic acid adapters are added analogously to the second extension round. The exposed sequences on the third permanent nucleic acid adapters are those exposed in the first round, meaning that subsequent extensions can be done by repeated and alternating addition of the same perm-adapters used in the second and third extension rounds.

Referring to FIG. 4G, for each extension, each mRNA generates a signal (such as a pseudo-color signal) corresponding to the FLASH-PAINT docking site added in that round. The resulting image thus consists of sequences of pseudo-colors which can be correlated to the mRNA sequence.

Referring to FIG. 4H, the first permanent nucleic acid adapters serve as a primer for reverse transcription of the mRNA. The resulting cDNA and adjacent perm-adapters are ligated together. Although some permanent nucleic acid adapters were added on the opposite strand (the antisense strand), their FLASH-PAINT docking site sequence is still encoded in its reverse complement on the other strand.

Referring to FIG. 4I, cDNA and ligated perm-adapters are isolated and sequenced, generated de novo sequencing information and identifying the sequence of added FLASH-PAINT docking sites, which can then be correlated to the appropriate sequence of pseudo-colors in the image.

In FIGS. 4A-4I, prime (′) is used to denote the reverse complement of a given sequence (e.g., (H1) base-pairs to (H1′)). The first permanent nucleic acid adapters consist of 3 regions: a 5′ poly-A region, a handle region (H1), and one of a set of readout sequences (e.g., DNA-PAINT docking sites) which transiently bind to a readout probes (e.g., DNA-PAINT imager or FLASH-PAINT adapter).

The poly-T region binds permanently to the poly-A tail of mRNA. The handle region enables the adapters to be iteratively appended to one another. The readout sequence enables the imaging and determination of the location of each of these probes.

By adding a cocktail of these probes (one probe for each readout sequence, all in equal concentration) to a cell, random readout sequences are attached to each mRNA.

After the sample is imaged, the next set of permanent nucleic acid adapters are added. The second set of permanent nucleic acid adapters consists of four regions: a handle region reverse-complementary to the previous round (H1′), a reverse-complement to one of the readout sequences, a second handle (H2′), and a second reverse-complement to a readout sequence (the identity of these two readout sequences are independent).

Since the exposed region of the previously attached first permanent nucleic acid adapters is H1 followed by one of the readout sequences, the newly added second permanent nucleic acid adapters are thus able to bind the exposed region permanently due to the additional length of base-pairing conferred by the handle sequence. The addition of the second permanent nucleic acid adapter exposes a new analogous region, H2′ followed by a reverse-complementary readout sequence, thereby blocking the previous readout sequence. The second round of imaging can then be done in the same manner as before.

The third round of permanent nucleic acid adapters consist of H2, a readout sequence, H1, and a second, independent readout sequence (analogous to the second permanent nucleic acid adapters). When the third permanent nucleic acid adapters bind the permanent adapters of the second round, a region of DNA consisting of H1 followed by a readout sequence is exposed. For an imaging stand point of view, the sample is at the same point as when the first round of extension has just been done.

By repeatedly adding the second and the third permanent nucleic acid adapters and imaging, a massive codebook of large number of possible barcodes can be generated.

Assuming that there have been k extension steps and n orthogonal readout sequences, up to n\hat{ }k random barcodes can be generated. When the number of the barcode combinations is sufficiently large, unique barcode for each mRNA molecule in a given sample can be expected. Because the barcodes were generated in a totally random manner, the image data (such as a sequence of pseudocolors for each molecule) contains no information about mRNA sequence or identity. However, because the permanent nucleic acid adapters are added permanently to the mRNA, the nucleic acid complex of the mRNA molecule and the barcoding adapters can be reverse-transcribed using the adapter as a primer and ligated together to become cDNA molecule suitable for sequencing. After isolating the cDNA for sequencing, the readout sequences will be sequenced along with their respective mRNA. Because the readout sequence is encoded within the DNA sequence, the sequencing data can be correlate to the sequence of pseudocolors in the image.

### Enumerated Embodiments

In some aspects, the present invention is directed to the following nonlimiting embodiments:

Embodiment 1: A method of identifying RNA molecules in a sample, the method comprising:
forming a randomized barcode on each of a plurality of RNA molecules in the sample, which comprises the steps of:
   attaching one by one in a stepwise manner, a plurality of permanent nucleic acid adapters to each of the plurality of RNA molecules, thereby forming a linear array of permanent nucleic acid adapters on each of the plurality of RNA molecules,
   wherein each permanent nucleic acid adapter in the linear array is selected randomly from a pool of different permanent nucleic acid adapters, and
   wherein each permanent nucleic acid adapter from the pool of different permanent nucleic acid adapters is associated with and identifiable by a detection motif;
introducing detection motifs into the sample, thereby associating the permanent nucleic acid adapter newly added in the linear array with the corresponding detection motifs;
acquiring imaging signals of the detection motifs in the sample;
determining the location of the RNA molecules in the sample and identifying the newly added permanent nucleic acid adapter based on locations and types of the detected imaging signals of the detection motifs;
combining the sequential arrangements of the permanent nucleic acid adapter in the linear arrays identified in the image acquisition steps into imaging-determined barcode information for each of the plurality of RNA molecules;
sequencing the plurality of RNA molecules, as well as the barcodes attached thereto to obtain RNA sequence for each of the plurality of RNA molecules and sequencing-determined barcode information associated with each of the plurality of RNA sequence; and
matching the RNA sequences obtained in the sequencing step to the imaging signals of the detection motifs in the sample by matching the imaging-determined barcode information with the sequencing-determined barcode information, thereby determining the location of each of the plurality of RNA molecules and the sequence thereof.

Embodiment 2: The method of Embodiment 1, wherein attaching the plurality of permanent nucleic acid adapters to each of the plurality of RNA molecules comprises, in each attaching step:
introducing in the sample the pool of different permanent nucleic acid adapters;
wherein one permanent nucleic acid adapter selected from the pool of permanent nucleic acid adapters is added to the RNA molecules or the permanent nucleic acid adapter already attached to the plurality of RNA molecules in an immediate prior attaching step, and
wherein each permanent nucleic acid adapter comprises:
   a hybridization region that hybridizes with, in a permanent manner, the RNA molecules or the permanent nucleic acid adapter added to the RNA molecules in the immediate prior round of addition; and
   one readout region selected from a predetermined group of readout regions, each associated with and identifiable by one detection motif.

Embodiment 3: The method of Embodiment 2, wherein introducing the detection motifs into the sample allows each of the newly attached permanent nucleic acid adapter to bind to one corresponding detection motif in a reversible manner via the readout region thereof.

Embodiment 4: The method of Embodiment 3, wherein the detection motifs reversibly bind to the corresponding permanent nucleic acid adapter via the bridging of a transient nucleic acid adapter.

Embodiment 5: The method of Embodiment 3, wherein
each detection motif is attached, optionally covalently, to a nucleic acid probe,
the detection motifs are introduced into the sample together with a pool of transient nucleic acid adapters,
each nucleic acid probe of the pool of transient nucleic acid adapters hybridizes with a corresponding transient nucleic acid adapter,
each transient nucleic acid adapter hybridizes with the readout region of a corresponding permanent nucleic acid adapter in a reversible manner.

Embodiment 6: The method of any one of Embodiments 1-5, wherein the sample is a fixed sample.

Embodiment 7: The method of any one of Embodiments 1-6, wherein the plurality of RNA molecules are a plurality of mRNA molecules, and wherein a first permanent nucleic acid adapter added in the first round of permanent nucleic acid adapter addition comprises:
a hybridization region that hybridizes with, in a permanent manner, the poly(A) tail of the mRNA molecules.

Embodiment 8: The method of any one of Embodiments 2-7, wherein at least one of the following applies:
(a) a dissociation constant (Kd) between the hybridization region of the permanent nucleic acid adapter and the plurality of RNA molecules, or a Kd between the hybridization region of the permanent nucleic acid adapter and the permanent nucleic acid adapter added in the immediate prior round of addition ranges from about 1000 fM to about 1 μM;
(b) an association rate constant (Kon) between the hybridization region of the permanent nucleic acid adapter and the RNA molecules, or a Kon between the hybridization region of the permanent nucleic acid adapter and the permanent nucleic acid adapter added in the immediate prior round of addition ranges from about 1*10³ 1/(M*sec)-1*10⁹ 1/(M*sec);
(c) a dissociation rate constant (Koff) between the hybridization region of the permanent nucleic acid adapter and the RNA molecules, or a Koff between the hybridization region of the permanent nucleic acid adapter and the permanent nucleic acid adapter added in the immediate prior round of addition ranges from about 1/2592000 1/sec to about 1/10 1/sec; or
(d) a number of complementary base pairs between the hybridization region of the permanent nucleic acid adapter and the RNA molecules, or a number of complementary base pairs between the hybridization region of the permanent nucleic acid adapter and the permanent nucleic acid adapter added in the immediate prior round of addition ranges from about 3 nt to about 250 nt.

Embodiment 9: The method of any one of Embodiments 4-8, wherein at least one of the following applies:
(a) a dissociation constant (Kd) between a permanent nucleic acid adapter and a corresponding transient adapter ranges from about 1 nM to about 1 mM;
(b) an association rate constant (Kon) between a permanent nucleic acid adapter and a corresponding transient adapter ranges from 1*10² 1/(M*sec) to about 1*10⁴ 1/(M*sec);
(c) a dissociation rate constant (Koff) between a permanent nucleic acid adapter and a corresponding transient adapter ranges from about 1/1000 (1/sec) to about 1 (1/sec); or
(d) a number of complementary base pairs between a permanent nucleic acid adapter and a corresponding transient adapter ranges from about 6 nt about 10 nt.

Embodiment 10: The method of any one of Embodiments 1-9, wherein forming the randomized barcodes on plurality of RNA molecules in the sample comprises:
(a) introducing to the sample a pool of first permanent nucleic acid adapters, thereby attaching each of the plurality of RNA molecules with one random first permanent nucleic acid adapter selected from the pool, wherein each first permanent nucleic acid adapter is associated with and identifiable by a corresponding detection motif;
(b) introducing to the sample a pool of sense-strand permanent nucleic acid adapters, thereby attaching the plurality of RNA molecules with one random sense-strand permanent nucleic acid adapter selected from the pool, wherein each sense-strand permanent nucleic acid adapter is associated with and identifiable by a corresponding detection motif; and
(c) introducing to the sample a pool of antisense-strand permanent nucleic acid adapters, thereby attaching the plurality of RNA molecules with one random antisense-strand permanent nucleic acid adapter selected from the pool, wherein each antisense-strand permanent nucleic acid adapter is associated with and identifiable by a corresponding detection motif,
wherein step (b)-(c) are performed one or more times in this order after step (a), and
wherein the randomized barcodes attached to each of the plurality of RNA molecules has the structure of: RNA-first permanent nucleic acid adapter-(antisense-strand permanent nucleic acid adapter-sense-strand permanent nucleic acid adapter) n, where n is an integer of 1 or larger.

Embodiment 11: The method of any one of Embodiments 1-10, wherein sequencing the plurality of RNA molecules and the barcodes attached thereto comprises:
subjecting nucleic acid complexes formed by the plurality of RNA molecules added with the plurality of permanent nucleic acid adapters to reverse transcription and ligation to form a plurality of corresponding cDNA molecules; and
sequencing the plurality of cDNA molecules.

Embodiment 12: The method of any one of Embodiments 1-11, wherein at least one of the following applies:
(a) the detection motifs are a fluorescence motif, optionally a fluorescent protein, a fluorescent small molecule, or a quantum dot;
(b) the detection motifs are a metal nanoparticle, optionally a gold nanoparticle;
(c) the detection motifs are a Raman scattering motif, optionally a Raman dye, optionally a Raman dye suitable for a stimulated Raman scattering microscopy; or
(d) the detection motifs are an isotope.

Embodiment 13: The method of any one of Embodiments 1-12, wherein at least one of the following applies:
(a) the sample is washed after a step of attaching one permanent nucleic acid adapters to remove excessive permanent nucleic acid adapters from the sample;
(b) the sample is not washed after a step of attaching a one permanent nucleic acid adapters;
(c) the sample is washed after a step of image acquisition to remove detection motifs; or
(d) the sample is not washed after a step of image acquisition.

Embodiment 14: The method of any one of Embodiments 1-13, wherein the sample is expanded according to an expansion microscopy technology.

Embodiment 15: A kit, comprising:
one or more pools of permanent nucleic acid adapters, wherein permanent nucleic acid adapters selected from the one or more pools of permanent nucleic acid adapters attach to RNA molecules one by one in a stepwise manner, thereby forming a barcode in the form of a linear array of permanent nucleic acid adapters; and
a pool of detection motifs, wherein each detection motif of the pool of detection motifs associates specifically with a readout region of a permanent nucleic acid adapter from each of the one or more pools of permanent nucleic acid adapter.

Embodiment 16: The kit of Embodiment 15, wherein
the one or more pools of permanent nucleic acid adapters comprises:
   a pool of first permanent nucleic acid adapters;
   a pool of antisense-strand permanent nucleic acid adapters; and
   a pool of sense-strand permanent nucleic acid adapters;
the permanent nucleic acid adapters from the pools of permanent nucleic acid adapters attach to RNA molecules one by one to form the barcode having the structure of: RNA-first permanent nucleic acid adapter-(antisense-strand permanent nucleic acid adapter-sense-strand permanent nucleic acid adapter)ₙ, where n is an integer of 1 or larger, and
wherein, in the barcode, each one of the first permanent nucleic acid adapter, the antisense-strand permanent nucleic acid adapter, and the sense-strand permanent nucleic acid adapter is one permanent nucleic acid adapter selected from the corresponding pools of one permanent nucleic acid adapters.

Embodiment 17: The kit of Embodiment 16, wherein each first permanent nucleic acid adapter of the pool of first permanent nucleic acid adapters comprises:
a hybridization region for permanently hybridizing with an RNA molecule; and
a readout region associated with an identifiable by a corresponding detection motif.

Embodiment 18: The kit of any one of Embodiments 16-17, wherein each antisense-strand permanent nucleic acid adapter of the pool of antisense-strand permanent nucleic acid adapters comprises:
a hybridization region for permanently hybridizing with some or all of the first permanent nucleic acid adapters or for permanently hybridizing with some or all of the sense-strand permanent nucleic acid adapters; and
a readout region associated with and identifiable by a corresponding detection motif.

Embodiment 19: The kit of any one of Embodiments 16-18, wherein each sense-strand permanent nucleic acid adapter of the pool of sense-strand permanent nucleic acid adapters comprises:
a hybridization region for permanently hybridizing with some or all of the antisense-strand permanent nucleic acid adapters; and
a readout region associated with and identifiable by a corresponding detection motif.

Embodiment 20: The kit of any one of Embodiments 16-19, wherein at least one of the following applies:
(a) a dissociation constant (Kd) between the first permanent nucleic acid adapter and the RNA molecules being barcoded, or a Kd between the antisense-strand permanent nucleic acid adapter and the adjacent first permanent nucleic acid adapter or the adjacent sense-strand permanent nucleic acid adapter in the barcode ranges from about 1000 fM to about 1 μM;
(b) an association rate constant (Kon) between the first permanent nucleic acid adapter and the RNA molecules being barcoded, or a Kon between the antisense-strand permanent nucleic acid adapter and the adjacent first permanent nucleic acid adapter or the adjacent sense-strand permanent nucleic acid adapter in the barcode ranges from about 1*10³ 1/(M*sec) to about 1*10⁹ 1/(M*sec);
(c) a dissociation rate constant (Koff) between the first permanent nucleic acid adapter and the RNA molecules being barcoded, or a Koff between the antisense-strand permanent nucleic acid adapter and the adjacent first permanent nucleic acid adapter or the adjacent sense-strand permanent nucleic acid adapter in the barcode ranges from about 1/2592000 1/sec to about 1/10 1/sec; or
(d) a number of complementary base pairs between the first permanent nucleic acid adapter and the RNA molecules being barcoded, or a Koff between the antisense-strand permanent nucleic acid adapter and the adjacent first permanent nucleic acid adapter or the adjacent sense-strand permanent nucleic acid adapter in the barcode ranges from about 3 nt to about 250 nt.

Embodiment 21: The kit of any one of Embodiments 15-19, wherein each detection motif is attached, optionally covalently, to a nucleic acid probe, and wherein the kit further comprises:
a pool of transient nucleic acid adapters,
wherein each nucleic acid probe of the pool of transient nucleic acid adapters hybridizes with a corresponding transient nucleic acid adapter, and
wherein each transient nucleic acid adapter hybridizes with the readout region of a corresponding permanent nucleic acid adapter in a reversible manner.

Embodiment 22: The kit of Embodiment 21, wherein at least one of the following applies:
(a) a dissociation constant (Kd) between a permanent nucleic acid adapter and a corresponding transient adapter ranges from about 1 nM to about 1 mM;
(b) an association rate constant (Kon) between a permanent nucleic acid adapter and a corresponding transient adapter tor ranges from about 1*10² 1/(M*sec) to about 1*10⁴ 1/(M*sec);
(c) a dissociation rate constant (Koff) between a permanent nucleic acid adapter and a corresponding transient adapter ranges from about 1/1000 (1/sec) to about 1 (1/sec); or
(d) a number of complementary base pairs between a permanent nucleic acid adapter and a corresponding transient adapter ranges from about 6 nt to about 10 nt.

Embodiment 23: The kit of any one of Embodiments 15-22, wherein at least one of the following applies:
(a) the detection motifs are a fluorescence motif, optionally a fluorescent protein, a fluorescent small molecule, or a quantum dot;
(b) the detection motifs are a metal nanoparticle, optionally a gold nanoparticle;
(c) the detection motifs are a Raman scattering motif, optionally a Raman dye, optionally a Raman dye suitable for a stimulated Raman scattering microscopy; or
(d) the detection motifs are an isotope.

Embodiment 24: A method of identifying RNA molecules in a fixed sample, the method comprising:
(a) contacting the sample with a first plurality of permanent adapters, wherein each permanent adapter in the first plurality of permanent adapters comprising a poly-T region, a first handlebar region and a readout sequence;
(b) contacting the sample with a first plurality of transient adapters and a first plurality of imager probes, wherein
   each transient adapter in the first plurality of transient adapters comprises a region complementary to the readout sequence on at least one of the permanent adapters in the first plurality of permanent adapters and an imager probe docking site;
   each imager probe of the first plurality of imager probes comprises a region complementary to the detector docking site on at least one of the transient adapters in the first plurality of transient adapters and a detection motif;
(c) imaging the sample to detect the detection motifs in the first plurality of imager probes;
(d) contacting the sample with a second plurality of permanent adapters, wherein each permanent adapter in the second plurality of permanent adapters comprises a region complementary to the first handlebar region as well as at least one readout sequence, a second handlebar region and a readout sequence;
(e) contacting the sample with a second plurality of transient adapters and a second plurality of imager probes, wherein
   each transient adapter in the second plurality of transient adapters comprising a region complementary to the readout sequence on at least one of the permanent adapters in the second plurality of permanent adapters and an imager probe docking site;
   each imager probe of the second plurality of imager probes comprises a region complementary to the detector docking site on at least one of the transient adapter adapters in the second plurality of transient adapters and a detection motif;
(f) imaging the sample to detect the detection motifs in the second plurality of imager probes;
(g) contacting the sample with a third plurality of permanent adapters, wherein each permanent adapter in the third plurality of permanent adapters comprises a region complementary to the second handlebar region as well as at least one readout sequence, a first handlebar region and a readout sequence;
(h) contacting the sample with a third plurality of transient adapters and a third plurality of imager probes, wherein
   each transient adapter in the third plurality of transient adapters comprising a region complementary to the readout sequence on at least one of the permanent adapters in the third plurality of permanent adapters and an imager probes docking site;
   each imager probe of the third plurality of imager probes comprises a region complementary to the detector docking site on at least one of the transient adapters in the third plurality of transient adapters and a detection motif;
(i) imaging the sample to detect the detection motifs in the third plurality of imager probes;
(j) determining the spatial locations of the RNA molecules according to the spatial locations of the detection motifs detected in steps (c), (f), and/or (i);
(k) combining all detection motifs detected in steps (c), (f), and (i) into imaging-determined barcode information for the RNA molecules;
(l) sequencing nucleic acid complexes comprising the RNA molecules and the permanent adapters attached thereon to obtain RNA sequences correlated with sequencing determined barcode information;
(m) correlating the spatial locations of the RNA molecules with the RNA sequences by correlating the imaging-determined barcode information with the sequencing determined barcode information,
wherein steps (a), (c), (d), (f), (g), and (i) are carried out in this order.

Embodiment 25: The method of Embodiment 24, wherein steps (d), (f), (g), (i) in claim 24 are repeated for one or more times before step (1).

Embodiment 26: The method of any one of Embodiments 24-25, wherein steps (b), (e) and (h) are carried out at the same time such that the first, second and third plurality of transient adapters and the first, second, and third plurality of imager probes are contacted with the sample as a mixture.

Embodiment 27: The method of Embodiment 26, wherein steps (b), (e) and (h) are carried out before, after, or at the same time with step (a).

Embodiment 28: A method of identifying molecules in a sample, the method comprising:
forming a randomized barcode on each of a plurality of molecules in the sample, which comprises the steps of:
   attaching one by one in a stepwise manner, a plurality of permanent nucleic acid adapters to each of the plurality of molecules, thereby forming a linear array of permanent nucleic acid adapters on each of the plurality of molecules,
   wherein each permanent nucleic acid adapter in the linear array is selected randomly from a pool of different permanent nucleic acid adapters, and
   wherein each permanent nucleic acid adapter from the pool of different permanent nucleic acid adapters is associated with and identifiable by a detection motif;
introducing detection motifs into the sample, thereby associating the permanent nucleic acid adapter newly added in the linear array with the corresponding detection motifs;
acquiring imaging signals of the detection motifs in the sample;
determining the location of the molecules in the sample and identifying the newly added permanent nucleic acid adapter based on locations and types of the detected imaging signals of the detection motifs;
combining the sequential arrangements of the permanent nucleic acid adapter in the linear arrays identified in the image acquisition steps into imaging-determined barcode information for each of the plurality of molecules;
identifying the plurality of molecules, as well as the barcodes attached thereto to; and
matching the molecule identities obtained in the identification step to the imaging signals of the detection motifs in the sample by matching the imaging-determined barcode information with the identified barcode information, thereby determining the location of each of the plurality of molecules and the identity thereof.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of identifying RNA molecules in a sample, the method comprising:
forming a randomized barcode on each of a plurality of RNA molecules in the sample, which comprises the steps of:
attaching one by one in a stepwise manner, a plurality of permanent nucleic acid adapters to each of the plurality of RNA molecules, thereby forming a linear array of permanent nucleic acid adapters on each of the plurality of RNA molecules,
wherein each permanent nucleic acid adapter in the linear array is selected randomly from a pool of different permanent nucleic acid adapters, and
wherein each permanent nucleic acid adapter from the pool of different permanent nucleic acid adapters is associated with and identifiable by a detection motif;
introducing detection motifs into the sample, thereby associating the permanent nucleic acid adapter newly added in the linear array with the corresponding detection motifs;
acquiring imaging signals of the detection motifs in the sample;
determining the location of the RNA molecules in the sample and identifying the newly added permanent nucleic acid adapter based on locations and types of the detected imaging signals of the detection motifs;
combining the sequential arrangements of the permanent nucleic acid adapter in the linear arrays identified in the image acquisition steps into imaging-determined barcode information for each of the plurality of RNA molecules;
sequencing the plurality of RNA molecules, as well as the barcodes attached thereto to obtain RNA sequence for each of the plurality of RNA molecules and sequencing-determined barcode information associated with each of the plurality of RNA sequence; and
matching the RNA sequences obtained in the sequencing step to the imaging signals of the detection motifs in the sample by matching the imaging-determined barcode information with the sequencing-determined barcode information, thereby determining the location of each of the plurality of RNA molecules and the sequence thereof.

2. The method of claim 1, wherein attaching the plurality of permanent nucleic acid adapters to each of the plurality of RNA molecules comprises, in each attaching step:
introducing in the sample the pool of different permanent nucleic acid adapters;
wherein one permanent nucleic acid adapter selected from the pool of permanent nucleic acid adapters is added to the RNA molecules or the permanent nucleic acid adapter already attached to the plurality of RNA molecules in an immediate prior attaching step, and
wherein each permanent nucleic acid adapter comprises:
a hybridization region that hybridizes with, in a permanent manner, the RNA molecules or the permanent nucleic acid adapter added to the RNA molecules in the immediate prior round of addition; and
one readout region selected from a predetermined group of readout regions, each associated with and identifiable by one detection motif.

3. The method of claim 2, wherein introducing the detection motifs into the sample allows each of the newly attached permanent nucleic acid adapter to bind to one corresponding detection motif in a reversible manner via the readout region thereof.

4. The method of claim 3, wherein the detection motifs reversibly bind to the corresponding permanent nucleic acid adapter via the bridging of a transient nucleic acid adapter.

5. The method of claim 3, wherein
each detection motif is attached, optionally covalently, to a nucleic acid probe,
the detection motifs are introduced into the sample together with a pool of transient nucleic acid adapters,
each nucleic acid probe of the pool of transient nucleic acid adapters hybridizes with a corresponding transient nucleic acid adapter,
each transient nucleic acid adapter hybridizes with the readout region of a corresponding permanent nucleic acid adapter in a reversible manner.

6. The method of claim 1, wherein the sample is a fixed sample.

7. The method of claim 1, wherein the plurality of RNA molecules are a plurality of mRNA molecules, and wherein a first permanent nucleic acid adapter added in the first round of permanent nucleic acid adapter addition comprises:
a hybridization region that hybridizes with, in a permanent manner, the poly(A) tail of the mRNA molecules.

8. The method of claim 2, wherein at least one of the following applies:
(a) a dissociation constant (Kd) between the hybridization region of the permanent nucleic acid adapter and the plurality of RNA molecules, or a Kd between the hybridization region of the permanent nucleic acid adapter and the permanent nucleic acid adapter added in the immediate prior round of addition ranges from about 1000 fM to about 1 μM;
(b) an association rate constant (Kon) between the hybridization region of the permanent nucleic acid adapter and the RNA molecules, or a Kon between the hybridization region of the permanent nucleic acid adapter and the permanent nucleic acid adapter added in the immediate prior round of addition ranges from about 1*10³ 1/(M*sec)-1*10⁹ 1/(M*sec);
(c) a dissociation rate constant (Koff) between the hybridization region of the permanent nucleic acid adapter and the RNA molecules, or a Koff between the hybridization region of the permanent nucleic acid adapter and the permanent nucleic acid adapter added in the immediate prior round of addition ranges from about 1/2592000 1/sec to about 1/10 1/sec; or
(d) a number of complementary base pairs between the hybridization region of the permanent nucleic acid adapter and the RNA molecules, or a number of complementary base pairs between the hybridization region of the permanent nucleic acid adapter and the permanent nucleic acid adapter added in the immediate prior round of addition ranges from about 3 nt to about 250 nt.

9. The method of claim 4, wherein at least one of the following applies:
(a) a dissociation constant (Kd) between a permanent nucleic acid adapter and a corresponding transient adapter ranges from about 1 nM to about 1 mM;
(b) an association rate constant (Kon) between a permanent nucleic acid adapter and a corresponding transient adapter ranges from 1*10² 1/(M*sec) to about 1*10⁴ 1/(M*sec);
(c) a dissociation rate constant (Koff) between a permanent nucleic acid adapter and a corresponding transient adapter ranges from about 1/1000 (1/sec) to about 1 (1/sec); or
(d) a number of complementary base pairs between a permanent nucleic acid adapter and a corresponding transient adapter ranges from about 6 nt about 10 nt.

10. The method of claim 1, wherein forming the randomized barcodes on plurality of RNA molecules in the sample comprises:
(a) introducing to the sample a pool of first permanent nucleic acid adapters, thereby attaching each of the plurality of RNA molecules with one random first permanent nucleic acid adapter selected from the pool, wherein each first permanent nucleic acid adapter is associated with and identifiable by a corresponding detection motif;
(b) introducing to the sample a pool of sense-strand permanent nucleic acid adapters, thereby attaching the plurality of RNA molecules with one random sense-strand permanent nucleic acid adapter selected from the pool, wherein each sense-strand permanent nucleic acid adapter is associated with and identifiable by a corresponding detection motif; and
(c) introducing to the sample a pool of antisense-strand permanent nucleic acid adapters, thereby attaching the plurality of RNA molecules with one random antisense-strand permanent nucleic acid adapter selected from the pool, wherein each antisense-strand permanent nucleic acid adapter is associated with and identifiable by a corresponding detection motif,
wherein step (b)-(c) are performed one or more times in this order after step (a), and
wherein the randomized barcodes attached to each of the plurality of RNA molecules has the structure of: RNA-first permanent nucleic acid adapter-(antisense-strand permanent nucleic acid adapter-sense-strand permanent nucleic acid adapter)ₙ, where n is an integer of 1 or larger.

11. The method of claim 1, wherein sequencing the plurality of RNA molecules and the barcodes attached thereto comprises:
subjecting nucleic acid complexes formed by the plurality of RNA molecules added with the plurality of permanent nucleic acid adapters to reverse transcription and ligation to form a plurality of corresponding cDNA molecules; and
sequencing the plurality of cDNA molecules.

12. The method of claim 1, wherein at least one of the following applies:
(a) the detection motifs are a fluorescence motif, optionally a fluorescent protein, a fluorescent small molecule, or a quantum dot;
(b) the detection motifs are a metal nanoparticle, optionally a gold nanoparticle;
(c) the detection motifs are a Raman scattering motif, optionally a Raman dye, optionally a Raman dye suitable for a stimulated Raman scattering microscopy; or
(d) the detection motifs are an isotope.

13. The method of claim 1, wherein at least one of the following applies:
(a) the sample is washed after a step of attaching one permanent nucleic acid adapters to remove excessive permanent nucleic acid adapters from the sample;
(b) the sample is not washed after a step of attaching a one permanent nucleic acid adapters;
(c) the sample is washed after a step of image acquisition to remove detection motifs; or
(d) the sample is not washed after a step of image acquisition.

14. The method of claim 1, wherein the sample is expanded according to an expansion microscopy technology.

15. A kit, comprising:
one or more pools of permanent nucleic acid adapters, wherein permanent nucleic acid adapters selected from the one or more pools of permanent nucleic acid adapters attach to RNA molecules one by one in a stepwise manner, thereby forming a barcode in the form of a linear array of permanent nucleic acid adapters; and
a pool of detection motifs, wherein each detection motif of the pool of detection motifs associates specifically with a readout region of a permanent nucleic acid adapter from each of the one or more pools of permanent nucleic acid adapter.

16. The kit of claim 15, wherein
the one or more pools of permanent nucleic acid adapters comprises:
a pool of first permanent nucleic acid adapters;
a pool of antisense-strand permanent nucleic acid adapters; and
a pool of sense-strand permanent nucleic acid adapters;
the permanent nucleic acid adapters from the pools of permanent nucleic acid adapters attach to RNA molecules one by one to form the barcode having the structure of: RNA-first permanent nucleic acid adapter-(antisense-strand permanent nucleic acid adapter-sense-strand permanent nucleic acid adapter)ₙ, where n is an integer of 1 or larger, and
wherein, in the barcode, each one of the first permanent nucleic acid adapter, the antisense-strand permanent nucleic acid adapter, and the sense-strand permanent nucleic acid adapter is one permanent nucleic acid adapter selected from the corresponding pools of one permanent nucleic acid adapters.

17. The kit of claim 16, wherein each first permanent nucleic acid adapter of the pool of first permanent nucleic acid adapters comprises:
a hybridization region for permanently hybridizing with an RNA molecule; and
a readout region associated with an identifiable by a corresponding detection motif.

18. The kit of claim 16, wherein each antisense-strand permanent nucleic acid adapter of the pool of antisense-strand permanent nucleic acid adapters comprises:
a hybridization region for permanently hybridizing with some or all of the first permanent nucleic acid adapters or for permanently hybridizing with some or all of the sense-strand permanent nucleic acid adapters; and
a readout region associated with and identifiable by a corresponding detection motif.

19. The kit of claim 16, wherein each sense-strand permanent nucleic acid adapter of the pool of sense-strand permanent nucleic acid adapters comprises:
a hybridization region for permanently hybridizing with some or all of the antisense-strand permanent nucleic acid adapters; and
a readout region associated with and identifiable by a corresponding detection motif.

20. The kit of claim 16, wherein at least one of the following applies:
(a) a dissociation constant (Kd) between the first permanent nucleic acid adapter and the RNA molecules being barcoded, or a Kd between the antisense-strand permanent nucleic acid adapter and the adjacent first permanent nucleic acid adapter or the adjacent sense-strand permanent nucleic acid adapter in the barcode ranges from about 1000 fM to about 1 μM;
(b) an association rate constant (Kon) between the first permanent nucleic acid adapter and the RNA molecules being barcoded, or a Kon between the antisense-strand permanent nucleic acid adapter and the adjacent first permanent nucleic acid adapter or the adjacent sense-strand permanent nucleic acid adapter in the barcode ranges from about 1*10³ 1/(M*sec) to about 1*10⁹ 1/(M*sec);
(c) a dissociation rate constant (Koff) between the first permanent nucleic acid adapter and the RNA molecules being barcoded, or a Koff between the antisense-strand permanent nucleic acid adapter and the adjacent first permanent nucleic acid adapter or the adjacent sense-strand permanent nucleic acid adapter in the barcode ranges from about 1/2592000 1/sec to about 1/10 1/sec; or
(d) a number of complementary base pairs between the first permanent nucleic acid adapter and the RNA molecules being barcoded, or a Koff between the antisense-strand permanent nucleic acid adapter and the adjacent first permanent nucleic acid adapter or the adjacent sense-strand permanent nucleic acid adapter in the barcode ranges from about 3 nt to about 250 nt.

21. The kit of claim 15, wherein each detection motif is attached, optionally covalently, to a nucleic acid probe, and wherein the kit further comprises:
a pool of transient nucleic acid adapters,
wherein each nucleic acid probe of the pool of transient nucleic acid adapters hybridizes with a corresponding transient nucleic acid adapter, and
wherein each transient nucleic acid adapter hybridizes with the readout region of a corresponding permanent nucleic acid adapter in a reversible manner.

22. The kit of claim 21, wherein at least one of the following applies:
(a) a dissociation constant (Kd) between a permanent nucleic acid adapter and a corresponding transient adapter ranges from about 1 nM to about 1 mM;
(b) an association rate constant (Kon) between a permanent nucleic acid adapter and a corresponding transient adapter tor ranges from about 1*10² 1/(M*sec) to about 1*10⁴ 1/(M*sec);
(c) a dissociation rate constant (Koff) between a permanent nucleic acid adapter and a corresponding transient adapter ranges from about 1/1000 (1/sec) to about 1 (1/sec); or
(d) a number of complementary base pairs between a permanent nucleic acid adapter and a corresponding transient adapter ranges from about 6 nt to about 10 nt.

23. The kit of claim 15, wherein at least one of the following applies:
(a) the detection motifs are a fluorescence motif, optionally a fluorescent protein, a fluorescent small molecule, or a quantum dot;
(b) the detection motifs are a metal nanoparticle, optionally a gold nanoparticle;
(c) the detection motifs are a Raman scattering motif, optionally a Raman dye, optionally a Raman dye suitable for a stimulated Raman scattering microscopy; or
(d) the detection motifs are an isotope.

24. A method of identifying RNA molecules in a fixed sample, the method comprising:
(a) contacting the sample with a first plurality of permanent adapters, wherein each permanent adapter in the first plurality of permanent adapters comprising a poly-T region, a first handlebar region and a readout sequence;
(b) contacting the sample with a first plurality of transient adapters and a first plurality of imager probes, wherein
each transient adapter in the first plurality of transient adapters comprises a region complementary to the readout sequence on at least one of the permanent adapters in the first plurality of permanent adapters and an imager probe docking site;
each imager probe of the first plurality of imager probes comprises a region complementary to the detector docking site on at least one of the transient adapters in the first plurality of transient adapters and a detection motif;
(c) imaging the sample to detect the detection motifs in the first plurality of imager probes;
(d) contacting the sample with a second plurality of permanent adapters, wherein each permanent adapter in the second plurality of permanent adapters comprises a region complementary to the first handlebar region as well as at least one readout sequence, a second handlebar region and a readout sequence;
(e) contacting the sample with a second plurality of transient adapters and a second plurality of imager probes, wherein
each transient adapter in the second plurality of transient adapters comprising a region complementary to the readout sequence on at least one of the permanent adapters in the second plurality of permanent adapters and an imager probe docking site;
each imager probe of the second plurality of imager probes comprises a region complementary to the detector docking site on at least one of the transient adapters in the second plurality of transient adapters and a detection motif;
(f) imaging the sample to detect the detection motifs in the second plurality of imager probes;
(g) contacting the sample with a third plurality of permanent adapters, wherein each permanent adapter in the third plurality of permanent adapters comprises a region complementary to the second handlebar region as well as at least one readout sequence, a first handlebar region and a readout sequence;
(h) contacting the sample with a third plurality of transient adapters and a third plurality of imager probes, wherein
each transient adapter in the third plurality of transient adapters comprising a region complementary to the readout sequence on at least one of the permanent adapters in the third plurality of permanent adapters and an imager probes docking site;
each imager probe of the third plurality of imager probes comprises a region complementary to the detector docking site on at least one of the transient adapters in the third plurality of transient adapters and a detection motif;
(i) imaging the sample to detect the detection motifs in the third plurality of imager probes;
(j) determining the spatial locations of the RNA molecules according to the spatial locations of the detection motifs detected in steps (c), (f), and/or (i);
(k) combining all detection motifs detected in steps (c), (f), and (i) into imaging-determined barcode information for the RNA molecules;
(l) sequencing nucleic acid complexes comprising the RNA molecules and the permanent adapters attached thereon to obtain RNA sequences correlated with sequencing determined barcode information;
(m) correlating the spatial locations of the RNA molecules with the RNA sequences by correlating the imaging-determined barcode information with the sequencing determined barcode information,
wherein steps (a), (c), (d), (f), (g), and (i) are carried out in this order.

25. The method of claim 24, wherein steps (d), (f), (g), (i) in claim 24 are repeated for one or more times before step (1).

26. The method of claim 24, wherein steps (b), (e) and (h) are carried out at the same time such that the first, second and third plurality of transient adapters and the first, second, and third plurality of imager probes are contacted with the sample as a mixture.

27. The method of claim 26, wherein steps (b), (e) and (h) are carried out before, after, or at the same time with step (a).

28. A method of identifying molecules in a sample, the method comprising:
forming a randomized barcode on each of a plurality of molecules in the sample, which comprises the steps of:
attaching one by one in a stepwise manner, a plurality of permanent nucleic acid adapters to each of the plurality of molecules, thereby forming a linear array of permanent nucleic acid adapters on each of the plurality of molecules,
wherein each permanent nucleic acid adapter in the linear array is selected randomly from a pool of different permanent nucleic acid adapters, and
wherein each permanent nucleic acid adapter from the pool of different permanent nucleic acid adapters is associated with and identifiable by a detection motif;
introducing detection motifs into the sample, thereby associating the permanent nucleic acid adapter newly added in the linear array with the corresponding detection motifs;
acquiring imaging signals of the detection motifs in the sample;
determining the location of the molecules in the sample and identifying the newly added permanent nucleic acid adapter based on locations and types of the detected imaging signals of the detection motifs;
combining the sequential arrangements of the permanent nucleic acid adapter in the linear arrays identified in the image acquisition steps into imaging-determined barcode information for each of the plurality of molecules;
identifying the plurality of molecules, as well as the barcodes attached thereto to; and
matching the molecule identities obtained in the identification step to the imaging signals of the detection motifs in the sample by matching the imaging-determined barcode information with the identified barcode information, thereby determining the location of each of the plurality of molecules and the identity thereof.
